(19) United States
(12) Patent Application Publication
(10) Pub. No.: US 2006/0127878 A1
(43) Pub. Date: Jun. 15, 2006

(54) Hydrogel preparation and process of manufacture thereof

(76) Inventors: David Henry Salomon, Murrumbeena (AU); Greg Guanghua Qiao, Doncaster (AU); Alan Yik Lun Kwok, Hawthorn (AU)

Correspondence Address: MORGAN LEWIS & BOCKIUS LLP, 1111 PENNSYLVANIA AVENUE NW, WASHINGTON, DC, 20004, US

(21) Appl. No.: 10/540,206

(22) PCT Filed: Dec. 17, 2003
(86) PCT No.: PCT/AU03/01680

§ 371 Date: Dec. 30, 2005

(30) Foreign Application Priority Data
Dec. 18, 2002 (AU) 2002953408
Dec. 18, 2002 (AU) 2002953409
May 14, 2003 (AU) 2003902305

(51) Int. Cl. C12Q 1/00

(52) U.S. Cl. 435/4; 204/450

(57) ABSTRACT

A polymeric hydrogel having a network of a macropores and micropores formed by copolymerizing at least one monomer having at least one double bond and at least one crosslinker having at least two double bonds in the presence of an organic additive forming a hydro-organic system with water, and uses thereof as separation media.

### TECHNICAL FIELD

The present invention relates to a separation medium comprising a hydrogel preparation consisting of macropores and micropores obtainable by using a hydro-organic solvent.

### BACKGROUND ART

Hydrogels for Separation Processes

In many applications of separation processes, it is desirable to have a porous matrix with good water compatibility and mechanical properties. In general, two broad classes of matrixes have been used. One general class is derived from water insoluble polymers by precipitation procedures such as Diffusion Induced Phase Separation (DIPS) and Thermally Induced Phase Separation (TIPS). These matrixes are relatively hydrophobic. A typical example is polysulphones membranes, which sometime require surface treatment or modification by physical adsorption of hydrophilic polymers (e.g. poly(vinyl alcohol)) to achieve satisfactory water wetting properties.

In many applications it is preferred to synthesize hydrogels from water-soluble monomers by incorporating crosslinking monomers into the polymer network. Typical examples are the range of hydrogels prepared by the free-radical co-polymerization of acrylamide and N,N′-methylenebisacrylamide. Such hydrogels are relative to DIPS and TIPS more hydrophilic and more stable since the hydrophilic groups are an integral part of the polymer structure. It is well accepted that the range of monomers suitable for the production of such hydrogels is rather limited, and is restricted to the requirement that both the monomer and the corresponding polymer need to be soluble in the polymerization solvent.

To address this limitation, several attempts have been made to prepare hydrogels by the bulk polymerization of monomers that produce water insoluble polymers. It is well accepted that the porosity of such gels is dependent upon total monomer concentration of the reaction mixture. For example, hydrogels with higher total monomer content will have a tighter network structure because of increased inter-penetration of polymer chains during network formation (Baker, J.; Hong, L.; Blanch, H.; Prausnitz, J. Macromolecules 1994, 27, 1446). As a result of this, and their high polymer content, hydrogels prepared in bulk are normally poor in mechanical strength (glassy and brittle), low in biocompatibility and water content, and possess a very limited pore size range. The absence of water in the synthesis environment of such hydrogels also makes subsequent solvent exchange with water difficult.

Polymerization-induced phase separation (PIPS) is a process in which an initially homogeneous solution of monomer and solvent becomes phase separated during the course of its polymerization. In hydrogel synthesis, PIPS can be induced by a number of factors: continuous increase in the fraction of molecules with high molecular weight, the unfavourable interactions between the polymer and other species in the reaction mixture, or the elasticity of the resultant polymeric network (Du\check{s}ek, K J. J. Polym. Sci. Polym. Symp. 1967, 16, 1289; Boots, H. M. J.; Kloosterboer, J. G.; Serbutoviez, C.; Touwslager, F. J. Macromolecules 1996, 29, 7683). Depending on the relative rates of the phase separation and the polymerization processes, PIPS can occur by the mechanism of nucleation-growth in the metastable region, or by spinodal decomposition in the multiphase coexisting region of the phase diagram (Eliçabe, G. E.; Larrondo, H. A.; Williams, R. J. J. Macromolecules 1997, 30, 6550; Eliçabe, G. E.; Larrondo, H. A.; Williams, R. J. J. Macromolecules 1998, 31, 8173).

In the homo-polymerizations of a mono-vinyl monomer, during the course of the reaction, because of the continuous increase in the fraction of polymer in the reaction mixture, PIPS can occur if the polymers formed in the reaction mixture are not miscible with the polymerization solvent. For example, PIPS occurs at ˜30% monomer conversion during the polymerization of a mixture composed of 30% 2-hydroxyethyl methacrylate and 70% water when the molecular weight of the resultant polymer is ˜300,000; and at ˜25% monomer conversion during the polymerization of a mixture composed of 20% acrylamide, 32.5% poly(ethylene glycol)-400 when the molecular weight of the resultant polymer is ˜10,000.

Miscibility in a multi-component system is governed by its Gibbs free energy of mixing (ΔGₘᵢₓ), which is a function of the enthalpies of mixing and the entropies of mixing between the various components in the mixture (ΔGₘᵢₓ=ΔHₘᵢₓ−TΔSₘᵢₓ). Because the enthalpy of mixing between two chemically different polymers is mostly positive, increases in the average molecular weight of the polymer solution will decrease the overall entropy of the system. It is also expected to decrease the miscibility of the polymerization mixture. This leads to the occurrence of PIPS at lower monomer conversions. For example, the onset of PIPS is at ˜1% monomer conversion during the polymerization of a mixture composed of 20% acrylamide, 32.5% poly(ethylene glycol)-400 when the molecular weight of the resultant polymer is ˜5,500,000. Polymer systems with higher average molecular weight will be less miscible than correspond systems with lower average molecular weight.

In a simplified gel formation process by the free radical co-polymerization of mono-vinyl monomer and multi-vinyl crosslinker, linear polymers are first formed in the solution during the fast propagation step, and later crosslinked with other molecules in close proximity by reaction through their pendent double bonds and additional monomer units (Stepto, R. F. T. “Non-linear polymerization, gelation and network formation, structure and properties”, in Stepto, R. F. T. (ed.) Polymer Networks 1998; London, Blackie Academic & Professional, 14-63). Therefore, in a gel formation process, the average molecular weight of the polymer solution increases with increasing monomer conversion because of the ongoing crosslinking reactions.

Because hydrogels are defined as a network with infinite molecular weight which reaches the macroscopic dimensions of the sample itself (Flory P. J. Principles of polymer science. New York: Cornell University Press, 1953 (Chapter IX)), polymers with very high molecular weight are produced in the reaction mixture prior to the formation of a gel network. Such polymers are therefore expected to undergo phase separations when the polymerization solvent is immiscible with their corresponding linear polymer analogues with high molecular weight.

Acrylamide hydrogels, for separation in zone electrophoresis, were introduced in 1959 (Raymond, Weintraub, Science 1959, 130, 711) and widely used as matrices for gels, and other electrophoretic operations. For example, one membrane-based electrophoresis technique (Gradiflow™ (Gradipore, Australia)) involves a fixed boundary preparative electrophoresis method (U.S. Pat. No. 5,650,055, U.S. Pat. No. 5,039,386 and WO 0013776) and utilizes a thin acrylamide hydrogel membrane with a defined pore size (D. B. Rylatt, M. Napoli, D. Ogle, A Gilbert, S. Lim, and C. H. Nair, J. Chromatog., A, 1999, 865, 145-153). However, despite its widespread popularity, there are several potential hazards and limitations which accompany the use of acrylamide hydrogel. For example, although the polymer is not toxic, exposure to the monomer and crosslinker at manufacture during preparation of the gel poses significant health concerns. In addition, residual and derivative chemical present in the gel may also pose potential health concern.

Currently, the pore size range of commercially available membranes is somewhat limited. For example, large pores suitable for DNA and RNA separations are not routinely available. It is well known that for an acrylamide hydrogel, although an increase in pore size can be achieved by decreasing the polymer content, the mechanical strength and integrity will also be decreased. The loss of gel rigidity places a practical limit on the accessible size separation range of a given material. In order to attempt to overcome these problems and to obtain matrices of higher porosity, Righetti (U.S. Pat. No. 5,785,832) and Uriel (U.S. Pat. No. 3,578,604) proposed polyacrylamide-agarose mixed-bed matrices. The matrix was obtained by a simultaneous but independent process of agarose and acrylamide gelification leading to an intertwining of the two polymers. The agarose used, however, is normally based on naturally occurring raw materials which often have associated chemical and structural impurities.

Righetti (U.S. Pat. No. 5,470,916) described a process for synthesising polyacrylamide matrixes with large pores. The process consists of adding, to the polymerization monomer mixture, hydrophilic polymers (e.g. polyethylene glycol, polyvinylpyrrolidone, hydroxymethyl cellulose) which, when added at a given concentration to the monomer mixture, force the chains to agglomerate together, thus forming a gel network having fibres of a much larger diameter than a regular acrylamide hydrogel. It was understood that the large pores were formed due to the competition between gelation and phase separation in the system (Asnaghi, D., Giglio, M., Bossi, A., Righetti, P. G., J. Mol. Strut. 1996, 38, 37). It is, however, hard to control the ranges of pore size obtainable using this technique.

Another approach to the synthesis of hydrogels with large pores is provided by template strategies (Beginn, U., Adv. Mater. 1998, 19, 16). This process resembles macroscopic metal casting processes in which templates preform the shapes of the pores like casting-cores are introduced into a liquid system and subsequently embedded by hardening of the solvent (i.e. polymerization). After removal of these cores from the surrounding matrix the shape of the voids that remain reflects the form of the templates.

Rill et al. (Rill, R. L., Locke, B. R., Liu, Y., Dharia, J., Van Winkle, D. L., Electrophoresis 1996, 17, 1304; Rill, R. L., Van Winkle, D. L., Locke, B. R., Anal. Chem. 1998, 70, 2433, Chakrapani, M., Van Winkle, D. H., Rill, R. L., Langmuir 2002, 18, 6449) reported templated acrylamide hydrogels as gel electrophoresis matrix and potential support for gel permeation chromatography. They showed that templating gels with sodium dodecyl sulfate (SDS) at concentrations up to 20% altered the electrophoretic separations of SDS-protein complexes in a manner consistent with the creation of pores by SDS micelles. Anderson (U.S. Pat. No. 5,244,799) described a process in which templated hydrogels were created by polymerizing a mixture of a hydrophilic monomer, polymerizing agent, an ionic surfactant and water. However, the usage of surfactants as template also have a few limitations, such as i) foaming problems during the degassing and the polymerization process; ii) the need to equilibrate the monomer solution (Method from Anderson involve the equilibration of the monomer solution for at least a week); iii) in such procedures, it is difficult to completely remove the ionic surfactant from the hydrogel after the polymerization step. Anderson described an additional step in which the hydrogel was to be treated with a non-ionic surfactant solution while Rill et al. reported the removal of 98% of SDS from the gel upon successive soaking in water. Residue ionic groups on the hydrogel matrix often caused undesirable electroendosmotic properties when exposed to an electric field, and more importantly, were able to affect biomolecule separation by physical interactions with charged groups on them; and iv) high surfactant concentrations are required to form the necessary interconnecting templating pores. At such concentrations, polyacrylamide is often incompatible with the ionic surfactant, resulting in undesirable phase separation during the polymerization. For example, Antonietti et al. (Antonietti, M., Caruso, R. A., Goltner, C. G., Weissenberger, M. C. Macromolecules 1999, 32 1383) reported during the formation of a variety of polymer gels such as polyacrylamide in the presence of lyotropic surfactant mesophases that “prior to polymerization all mixtures are transparent, and become opaque or turbid-white shortly after the start of the reaction”. Rill also reported that gels formed in the presence of 30% or more SDS became uniformly white as the surfactants were removed.

Undesirable swelling or shrinking has always been a drawback in the use of acrylamide hydrogels in non-aqueous operating systems such as the separation of ions in non-aqueous systems and the electrophoretic separation of hydrophobic proteins using organic solvents. Hydrogels synthesised in a solvent similar to that of its final operating environment will be more tolerant to solvent compositional changes. Typical solvents used in non-aqueous operating systems include alcohols, glycols, dimethyl formamide (DMF), dimethyl sulfoxide (DMSO), tetramethylurea, formamide, tetramethylene sulfone, chloral hydrate N-methyl acetamide, N-methyl pyrollidone and phenol. It is, however, well known that when amounts of water-miscible solvents such as DMF, DMSO, TMU, ethylene glycol, or propylene glycol are added to the acrylamide polymerization mixture, the mechanical strength and clearness of the polymer gel are severely compromised.

Amphiphilic polymer networks of α,ω-(meth)acryloyloxy monomers such as poly(2-hydroxyethyl methacrylate) (poly(HEMA) have been studied extensively as materials for pharmaceutical and biomedical applications, including carriers for controlled drug delivery and materials for prosthetic devices. The mechanical strength provided by the hydrophobic backbone and the hydrophilicity of the hydroxy and ester groups on the polymer side chains make polymers produced from HEMA excellent candidates for hydrogels for separation processes. Zewert and Harrington (U.S. Pat. No. 5,290,411; U.S. Pat. No. 5,290,411; Zewert, T., Harrington, M., Electrophoresis 1992, 13, 817-824; Zewert, T., Harrington, M., Electrophoresis 1992, 13, 824), and Solomon et al. (PCT AU01/01632) have described the usage of hydrogels prepared from α,ω-(meth)acryloyloxy monomers in various electrophoretic operations.

Most existing 2-Hydroxyethyl methacrylate (HEMA) systems are prepared in bulk, or with <50% diluent. Owing to the hydrophobicity of the network, organic diluents such as ethylene glycol and di(ethylene glycol) are normally used (WO 00/44356; Caliceti, P., Veronese, F., Schiavon, O., Il Farmaco 1992, 47, 275, Carenza, M., Radiat. Phys. Chem. 1993, 42, 897). Although the properties of these hydrogels can be modified by crosslinking or by the use of different diluents, their swelling in water is thermodynamically limited to ˜40% (Havsky, M., Prins, W., Macromolecules 1970, 3, 415; Nakamura, K., Nakagawa, T., Journal of Polymer Science 1975, 13, 2299).

As a result, such HEMA hydrogels are normally poor in mechanical strength (glassy and brittle), low in biocompatibility, low in water content, and possess a very limited pore size range. The absence of water in the synthesis environment of such hydrogels also made subsequent solvent exchange with water difficult. In addition, the toxicity of some of the diluents is of great concern. Such hydrogels have been predominantly used in applications that desire low water swelling, such as contact lenses and transport membranes for gases and ions (Corkhill, P. H., Jolly, A. M., Ng, C. O., Tighe, B. J. Polymer 1987, 28, 1758; Hamilton, C. J., Murphy, S. M., Atherton, N. D., Tighe, B. J., Polymer 1988, 29, 1879).

It is well accepted that the porosity of such hydrogels is dependent upon the particular monomer, particular crosslinking agent, and the degree of crosslinking. For example, hydrogels with higher total monomer content will have a tighter network structure because of increased interpenetration of polymer chains during network formation (Baker, J.; Hong, L.; Blanch, H.; Prausnitz, J. Macromolecules 1994, 27, 1446). It is thus highly desirable to be able to produce an HEMA hydrogel with high water content at a low initial concentration of monomers (<50 wt %) in order to obtain the desired biocompatibility and pore sizes for applications such as electrophoresis separation membranes.

Several attempts have been made to improve the water swelling properties of HEMA hydrogels and to prepare such gel at a low initial concentration of monomers.

i) HEMA hydrogels were synthesised in various hydro-organic solvents. Refojo (Refojo, M., Journal of Polymer Science: Part A-1 (1967), 5, 3103) reported that visually clear hydrogels of poly(2-hydroxyethyl methacrylate) may be prepared by conducting the polymerization in ethylene glycol-water solution. The phase separation limit for this type of system was reported to be 45% of water in the reaction solution, allowing the total monomer concentrations to be decreased by the replacement of monomers with diluent (Warren, T., Prins, W., Macromolecules (1972), 5, 506). In addition to the fact that HEMA hydrogels prepared in such diluent were reported to exhibit a narrow range of swelling at equilibrium in water (41% water) regardless of the initial dilution of the monomer solution and relatively low level of crosslinking. Results from our laboratory have shown that this separation limit is highly dependent upon both the amount of crosslinker and the choices of diluent in the reaction solution, with some formulations forming heterogeneous opaque polymer mass even when the water content is below 45%. Zewert and Harrington (Zewert, T., Harrington, M., Electrophoresis 1992, 13, 817) reported HEMA hydrogel synthesis in aqueous sulfolane solution and concluded that HEMA polymerization is thoroughly incompatible with sulfolane even if sulfolane concentrations are as low as 10%.

ii) Various HEMA derivatives such as the poly(alkylene glycol) esters of acrylic or methacrylic acid (e.g. poly(ethylene glycol) methacrylate) were used instead of HEMA to prepare hydrogels with improved water swelling properties. The disadvantages of such monomers is that they are expensive and difficult to prepare. In addition, the pore size of hydrogels prepared by these monomers is also limited because of their large molecular weight, restricting the number of monomer units available in the monomer mixture.

iii) In order to obtain HEMA hydrogels with improved water swelling properties, it is common to copolymerize HEMA with a hydrophilic monomer such as acrylamide. Bajpai and Shrivastava (Bajpai, A. K., Shrivastava, M. J. Biomater. Sci. Polymer Edn 2002, 13, 237) copolymerised HEMA with acrylamide (% acrylamide>40 mol %) in the presence of a hydrophilic polymer, poly(ethylene glycol) (PEG, MW 600). It was found that the swelling ratio of such hydrogel increases with increasing PEG 600 content in the monomer mixture to a maximum at 4.31% (by weight). Such hydrogels, according to the authors, “could be regarded as a network of poly(ethylene glycol) and poly(HEMA-co-acrylamide) chains thus creating free volumes of varying meshes for accommodating penetration of water molecules”. It was also stated by Baipai and Shrivastava that there is no clear advantage of using a highly hydrophilic polymer content—“beyond 0.56 of PEG (600) content (4.31%), the network density of the gel may became so high that mesh sizes of free volumes available between the network chains get reduced . . . thus decreasing the swelling of the gel”. It is clear that the co-polymerization of acrylamide with HEMA does not eliminate the disadvantages associated with acrylamide hydrogels.

The present inventors have now developed new hydrogels suitable for a number of separation techniques. The present invention also provides visually clear hydrogels with good water compatibility and swelling properties to be synthesized from monomers in hydro-organic or organic solvents.

### DISCLOSURE OF INVENTION

In a first aspect, the present invention provides a process for producing a polymeric hydrogel having a network containing macropores and micropores, the process comprising:

(a) forming a mixture by adding at least one monomer having at least one double bond, at least one crosslinker having at least two double bonds, an initiation system, and an organic additive to form a hydro-organic system with water; and

(b) allowing the monomer and crosslinker to copolymerize to form a hydrogel having a polymeric network containing macropores and micropores.

The monomer having at least one double bond may be selected from polyol esters of acrylic or methacrylic acid, where the polyol is selected from a group which includes polyethylene glycol, a range of polyethylene glycol esters or ethers, polypropylene glycol, a range of polypropylene glycol esters or ethers, random or block copolymers of ethylene glycol and propylene glycol, or any suitable polyols such as glycerol, pentaerythritol, ethylene glycol or propylene glycol which are fully or partly esterified. Mixtures consist of at least two of the above monomers can also be used.

Mixtures of the above monomer with any other well-known monomers suitable for free radical polymerization may be used.

Preferably the monomer is used from about 1 to 80%, more preferably, from about 5 to 50%.

Preferably, the monomer is one or more hydrophilic monomers from the esters of acrylic or methacrylic acids.

In one preferred form, the monomer is hydroxyethyl methacrylate (HEMA).

The crosslinker having at least two double bond may be selected from esters of acrylic and/or methacrylic acid, or acrylic or methacrylic acid with various polyols. Typical polyols include polyethylene glycol, a range of polyethylene glycol, a range of polypropylene glycol, random or block copolymers of ethylene glycol and propylene glycol, or any suitable polyols such as glycerol, pentaerythritol, ethylene glycol or propylene glycol which may be partly esterified (for example, glycerol can be esterified with two molecules of methacrylic acid to give the crosslinking mixture). Mixtures consist of at least two of the above crosslinkers can also be used.

Mixtures of above crosslinker with any other well-known crosslinkers suitable for free radical polymerization may be used.

Preferably use of the above crosslinker with greater than about 50% in the mixture of crosslinkers; more preferably greater than about 80%.

In one preferred form, the crosslinker is ethylene glycol dimethacrylate (EGDMA).

Preferably, the polymeric hydrogel is made from a mixture of monomer content of about 10 to 40%M and crosslinker of about 1 to 30%X before polymerization. When HEMA and EGDMA are used, the preferred compositions of monomer mixture of HEMA with EGDMA are less than about 40% M and less than about 20%X, respectively. It will be appreciated, however, that other concentrations can be used depending on the monomer and crosslinker used.

Any suitable free radical producing method can be used as the initiation system. The initiation system is preferably formed by the redox, thermal or photo initiator/s. More preferably, the redox initiator is formed by ammonium persulphate (APS) with N,N,N′,N′-tetramethylethylenediamine (TEMED).

The organic additive, which may be monomeric or polymeric (such as ethylene glycol or polyethylene glycol), is preferably a hydrophilic polymer miscible with water and miscible with a linear polymer produced from the monomer used for copolymerization; or a hydrophilic polymer miscible with water and has a similar solubility parameter (±10(MPa)^{0.5}) to that of a polymer produced from the monomer used for copolymerization. The organic additive can be a single entity acting as both a porogen to form macropores during the polymerization and a solvent with water to form the hydro-organic solvent.

The organic additive is preferably selected from ethylene glycol or polyethylene glycol, propylene glycol or polypropylene glycol, random or block copolymers of any of the above mixtures, or any of the above additives that have an ester or ether end group. Mixtures consist of at least two of the additives can also be used.

More preferably, the organic additive has the following general formulation:

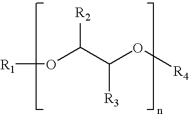

R₁, R₄=H, CH₃, —(CH₂)ₓ—CH₃ (x=1-4), —C(═O)—R₅ (R₅=(CH₂)ₓ—CH₃ (x=0-4))

R₂, R₃=H, CH₃, —(CH₂)ₓ—CH₃ (x=1-4), OH

In a preferred from, the organic additive is a polyethylene glycol or polypropylene glycol. The polyethylene glycol preferably has a molecular weight range from about 100 to 100000; preferably from about 200 to 10000; more preferably from about 400 to 4000.

The polypropylene glycol typically has a molecular weight range from about 100 to 100000; preferably from 200 to 10000; more preferably from about 58 to 600.

In another preferred form, the organic additive is a copolymer with a hydrophilic component and a hydrophobic component. Preferably, the organic additive is a copolymer of polyethylene glycol with polypropylene glycol.

In use, the polymeric hydrogel formed can be used in the hydro-organic solvent or the hydro-organic solvent components exchanged with water.

In a second aspect, the present invention provides a polymeric hydrogel having a network containing macropores and micropores produced by the process according to the first aspect of the present invention.

In a third aspect, the present invention provides a polymeric hydrogel comprising a network of macropores and micropores formed by copolymerizing at least one monomer having at least one double bond and at least one crosslinker having at least two double bonds in the presence of a organic additive forming a hydro-organic system with water.

The monomer having at least one double bond may be selected from polyol esters of acrylic or methacrylic acid, where the polyol is selected from a group which includes polyethylene glycol, a range of polyethylene glycol esters or ethers, polypropylene glycol, a range of polypropylene glycol esters or ethers, random or block copolymers of ethylene glycol and propylene glycol, or any suitable polyols such as glycerol, pentaerythritol, ethylene glycol or propylene glycol which are fully or partly esterified. Mixtures consist of at least two of the above monomers can also be used.

Mixtures of the above monomer with any other well-known monomers suitable for free radical polymerization may be used.

Preferably use of above monomer with greater than 50% in the mixture of monomers; more preferably greater than 80%.

Preferably, the monomer is one or more hydrophilic monomers from the esters of acrylic or methacrylic acids.

In one preferred form, the monomer is hydroxyethyl methacrylate (HEMA).

The crosslinker having at least two double bond may be selected from esters of acrylic and/or methacrylic acid, or acrylic or methacrylic acid with various polyol. Typical polyols are polyethylene glycol, a range of polyethylene glycol, a range of polypropylene glycol, random or block copolymers of ethylene glycol and propylene glycol, or any suitable polyols such as glycerol, pentaerythritol, ethylene glycol or propylene glycol which may be partly esterified (for example, glycerol can be esterified with two molecules of methacrylic acid to give the crosslinking mixture). Mixtures consist of at least two of the above crosslinkers can also be used.

Mixtures of above crosslinker with any other well-known crosslinkers suitable for free radical polymerization may be used.

Preferably use of the above crosslinker with greater than 50% in the mixture of crosslinkers; more preferably greater than 80%.

In one preferred form, the crosslinker is ethylene glycol dimethacrylate (EGDMA).

Preferably, the polymeric hydrogel is made from a mixture of monomer content of about 10 to 40%M and crosslinker of about 1to 30%X before polymerization. When HEMA and EGDMA are used, the preferred compositions of monomer mixture of HEMA with EGDMA are less than about 40% M and less than about 20% X. It will be appreciated, however, that other concentrations can be used depending on the monomer and crosslinker used.

Any suitable free radical producing method can be used as the initiation system. The initiation system is preferably formed by the redox, thermal or photo initiator/s. More preferably, the redox initiator is formed by ammonium persulphate (APS) with N,N,N′,N′-tetramethylethylenediamine (TEMED).

The organic additive, which may be monomeric or polymeric, is preferably a hydrophilic polymer miscible with water and miscible with a linear polymer produced from the monomer used for copolymerization; or a hydrophilic polymer miscible with water and has a similar solubility parameter (±10(MPa)^{0.5}) to that of a polymer produced from the monomer used for copolymerization. The organic additive can be a single entity acting as both a porogen to form macropores during the polymerization and a solvent with water to form the hydro-organic solvent.

The organic additive is preferably selected from ethylene glycol or polyethylene glycol, propylene glycol or polypropylene glycol, random or block copolymers of any of the above mixtures, or any of the above additives that have an ester or ether end group. Mixtures consist of at least two of the additives can also be used.

More preferably, the organic additive has the following general formulation:

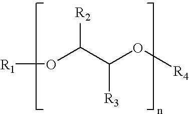

R₁, R₄═H, CH₃, —(CH₂)ₓ—CH₃ (x=1-4), —C(═O)—R₅(R₅═(CH₂)ₓ—CH₃ (x=0-4))

R₂, R₃H, CH₃, —(CH₂)ₓ—CH₃ (x=1-4), OH

In a preferred from, the organic additive is a polyethylene glycol or polypropylene glycol. The polyethylene glycol preferably has a molecular weight range from about 100 to 100000; preferably from about 200 to 10000; more preferably from about 400 to 4000.

The polypropylene glycol typically has a molecular weight range from about 100 to 100000; preferably from 200 to 10000; more preferably from about 58 to 600.

In another preferred form, the organic additive is a copolymer with a hydrophilic component and a hydrophobic component. Preferably, the organic additive is a copolymer of polyethylene glycol with polypropylene glycol.

Preferably, the mixture is degassed to remove any dissolved oxygen prior to polymerization.

In use, the polymeric hydrogel formed can be used in the hydro-organic solvent or the hydro-organic solvent components exchanged with water.

In a fourth aspect, the present invention provides a separation medium formed from the polymeric hydrogel according to the second or third aspects of the present invention.

Preferably, the separation medium is in the form of membrane, slab, beads or column. The medium is particularly suitable as an electrophoretic medium capable of separating large biomolecules or compounds having a molecular weight of at least 2000 k.

In a fifth aspect, the present invention provides a visually clear polymeric hydrogel according to the second or thirds aspects of the present invention.

The present inventors have found that by the use of mixtures of water and water-miscible entities as the polymerization solvent, visually clear hydrogels can be prepared even when the polymerization solvent is immiscible with the corresponding linear polymer analogues. For example, a mixture of 20% poly(acrylamide)-5,500,000, 1% poly(vinyl alchol)-18,000 (88% hydrolyzed), and 79% water is immiscible, but the polymerization of 20% solutions of acrylamide and N,N′-methylenebisacrylamide can give visually clear gels; a mixture of 15% poly(2-hydroxyethyl methacrylate)-300,000, 75% ethylene glycol dimethyl ether or 75% poly(ethylene glycol) dimethyl ether, and 10% water is immiscible, but the polymerization of 15% solutions of 2-hydroxyethyl methacrylate and ethylene glycol dimethacrylate in these solvents can give visually clear gels.

These results are new and unexpected because the general teaching from most scientific literature on monomer selection for hydrogel synthesis is that the polymerization solvent should be a solvent for the linear analogues of the resultant polymeric network.

By the selection of the water-miscible entities, the ‘freezing point’ of the reaction mixture can be controlled such that it occurs at a monomer conversion lower than the critical monomer conversion for the onset of PIPS. The ‘freezing point’ of the reaction mixture is defined as the critical monomer conversion at which the viscosity of the mixture reaches a specific level when the mobility of polymer chains in the mixture becomes negligible and the dynamic concentration fluctuations of pre-gel polymer solutions are frozen in the final network structure. The resultant hydrogels of these systems will be visually clear and have a relatively uniform network because the polymer mixture was frozen in its miscible state before phase separation could occur. Hydrogels prepared by this approach have superior swelling, opitcal, and mechanical properties to that prepared by systems that reaches the phase boundary before the gel point. Those gels are formed from dispersions of precipitated polymers in the liquid phase (Okay O. Polymer 1999, 40, 4117) and are highly opaque polymer masses that have very different properties from hydrogels synthesized using our approach.

In a sixth aspect, the present invention provides a method for separating one or more compounds according to size using electrophoresis, the method comprising:

(a) providing a medium in the form of polymeric hydrogel having a network containing macropores and micropores according to the second or third aspects of the present invention;

(b) adding one or more compounds to part of the medium; and

(c) applying an electric potential causing at least one compound to pass through the medium, wherein movement through the medium is related to the size of the compound.

In a seventh aspect, the present invention provides a size exclusion electrophoresis system comprising:

(a) a cathode;

(b) an anode; and

(c) a separation medium in the form of polymeric hydrogel having a network containing macropores and micropores according to the second or third aspects of the present invention capable of separating a mixture of compounds according to size, the medium disposed between the anode and cathode.

In a preferred form, the system further includes means for supplying a sample containing one or more compounds to be separated to the system.

In a preferred form, the system further includes means for retaining or capturing a compound separated by the system.

In a preferred form, the system further includes a voltage supply and means for applying an electric potential between the cathode and anode.

The system can be formed by having the separation medium disposed between two ion-permeable barriers forming two chambers either side of the size exclusion medium. Sample containing the compound(s) to be separated can be placed in one of the chambers and, under the influence of the applied voltage, a compound will move through the separation medium in accordance with its size (large molecules elute out first) to the second chamber where it can be retained or collected. It is also possible to have a plurality of different separation media disposed between the cathode and anode. In this form, preferably each separation medium would have a different pore structure so as to be able to separate compounds of different size.

In a eighth aspect, the present invention provides use of a separation medium in the form of polymeric hydrogel having a network containing macropores and micropores according to the second or third aspects of the present invention in size exclusion electrophoresis.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the present invention may be more clearly understood, preferred forms will be described with reference to the following drawings and examples.

### BRIEF DESCRIPTION OF THE DRAWINGS

FIG. 1 shows migration ratios of Kaleidoscope Pre-stained Standards in 10%M 2%X acrylamide gel cassette synthesized in water, aqueous solutions of ethylene glycol (25%) or propylene glycol (25%).

FIG. 2 shows migration ratios of SDS-PAGE Molecular Weight Standards (board range) in 10%M 2%X acrylamide gel cassette synthesized in water, or aqueous solutions of poly(ethylene glycol).

FIG. 3 shows migration ratios of SDS-PAGE Molecular Weight Standards (board range) in 10%M 2%X acrylamide gel cassette synthesized in water or aqueous solutions of tri(ethylene glycol) and poly(ethylene glycol).

FIG. 4 shows migration ratios of Kaleidoscope Prestained Standards in 10%M 2%X acrylamide gel cassette synthesized in water and aqueous solutions of tri(ethylene glycol).

FIG. 5 shows turbidity results of polymers synthesized according to Example 29.

FIG. 6 shows turbidity results of polymers synthesized according to Example 30.

FIG. 7 shows turbidity results of polymers synthesized according to Example 31.

FIG. 8 shows turbidity results of polymers synthesized according to Example 32.

FIG. 9 shows turbidity results of polymers synthesized according to Example 33.

FIG. 10 shows turbidity results of polymers synthesized according to Example 34.

FIG. 11 shows turbidity results of polymers synthesized according to Example 35.

FIG. 12 shows turbidity results of polymers synthesized according to Example 36.

FIG. 13 shows turbidity results of polymers synthesized according to Example 37.

FIG. 14 shows turbidity results of polymers synthesized according to Example 38.

FIG. 15 shows the separation and migration pattern of Bovine serum albumin (MW 67,000) by a 15%M 4%X HEMA/EGDMA membrane synthesized in 80% aqueous PEG 200 solution (Example 41) using 40 mM MES bis-TRIS buffer.

FIG. 16 shows turbidity results of polymers synthesized according to Example 56.

FIG. 17 shows a schematic diagram of the formation process of 20%M acrylamide hydrogels in the presence of water and a water-soluble entity. Line E represents systems with 0%X; line F, 2%X; line G, 3%X; line H, 10%X.

FIG. 18 shows real-time viscosity measurements of the polymerization of 20%M acrylamide solutions, in the presence of 17.5% PEG-400, at various %X. Time at which phase separation was observed in the samples are represented by dark coloured points (circle).

FIG. 19 shows turbidity measurements of 20%M 2%X acrylamide hydrogels synthesized in the presence of various amounts of PEG-400.

FIG. 20 shows the critical propylene glycol concentrations for the formation of visually hydrogels at various %M and %X.

FIG. 21 shows real-time viscosity measurements of the polymerization of 20%M 2%X HEMA solutions in the presence of various amounts of propylene glycol. Times at which phase separation was observed in the samples are represented by dark coloured points (circle)

FIG. 22 shows SEM images (10,000×) of cross-sectional interior of swollen 10%M 2%X acrylamide hydrogels synthesized in water (A), 50% ethylene glycol solution (B), and 50% propylene glycol solution (C).

### MODE(S) FOR CARRYING OUT THE INVENTION

Novel formulations for HEMA hydrogel synthesis

The present inventors have developed a new synthesis method using a mixture of water and water-miscible entities as the polymerization solvent such that HEMA hydrogels can be crosslinked with ethylene glycol dimethacryate (EGDMA) using low initial monomer content (5-50%). Using water-miscible entities such as polymers with repeating ethoxylated and propyoxylated units (e.g. poly(ethylene glycol) and poly(propylene glycol) or random or block copolymers of poly(ethylene glycol) at a polymeric-additive glycol-water ratio of about 9:1 to 1:9), hydrogels based on HEMA were successfully formed having higher water swelling properties and bigger pore sizes than those produced previously. Such hydrogels can be subsequently used as synthesized or after the water-miscible entities have been displaced with water. This result is unexpected, given that it is well known that high concentrations of hydrophilic polymer (i.e. poly(ethylene glycol) and poly(propylene glycol)) in acrylamide hydrogel synthesis would lead to phase separation of the reaction mixture. For example, Righetti (Righetti, P. G Chromatogr. A 1995, 698, 3) observed that when acrylamide hydrogels were synthesised in the presence of PEG 2000-20,000, turbid gels (phase separation) were produced and was a function of both length and concentration of the polymer. It was observed that longer polymer chains induce phase separation at lower concentration; all gels become turbid when the PEG concentration in the solvent exceed 10 wt %.

It was also discovered by the present inventors that as the molecular weight of the water-miscible entities increases, the pore size of the hydrogels becomes dependent upon the properties of the entities, with the entities acting as a “template”. In high molecular weight solvents, hydrogels synthesized in solutions of high molecular weight entities were observed to swell more than that of lower molecular weight. To our knowledge, this is the first system in which the templating system is also acting as the solvent for the hydrogel.

Multimodal hydrogels

Utilising the templating and the solvent properties of the water-miscible entities, it was discovered that multimodal HEMA hydrogels can be obtained by careful selection of the concentrations of monomer, the crosslinking extent, and the types and concentrations of water-miscible entities in a one-step process. Two general types of pores exist in such membranes—macropores formed by the template, and micropores formed by the crosslinking of polymer chains. Dependent upon the concentrations of the water-miscible entities, the macropores in the hydrogel can be continuous (i.e. interconnected), or non-continuous.

Derivatives of monomers such as the poly(alkylene glycol) esters of acrylic or methacrylic acid can also be used in the same manner as HEMA to prepare hydrogels with multimodal channels.

Such hydrogels are different from these synthesised by Zewert and Harrington (U.S. Pat. No. 5,290,411 and U.S. Pat. No. 5,290,411) because:

i) Their teaching indicates that the pore size of the gel is dependent upon the types and concentration of monomer and crosslinkers. Pore sizes of hydrogels according to the present invention are not only dependent upon the types and concentration of monomer and crosslinkers but also dependent upon the size of the water-miscible entities;

ii) The present hydrogels have two types of pores within its network, macropores and micropores;

iii) In the patent of Zewert and Harrington, organic solvents were added mainly for the usage of the resultant gel in organic electrophoresis and were not subsequently replaced with water. In the present invention, the water-miscible entities are acting both as a solvent and a template, and are subsequently exchanged with water.

Applications

HEMA hydrogels made with the above formulations are particularly well-suited for use as separation membranes for biomolecules. Other related areas of interest include biocompatible applications such as prosthetic devices, drug release matrixes, and tissue scaffolds.

Membrane-Based Electrophoresis

A number of membrane-based electrophoresis apparatus developed by Gradipore Limited, Australia were used in the following experiments. In summary, the apparatus typically included a cartridge which housed a number of membranes forming two chambers, cathode and anode connected to a suitable power supply, reservoirs for samples, buffers and electrolytes, pumps for passing samples, buffers and electrolytes, and cooling means to maintain samples, buffers and electrolytes at a required temperature during electrophoresis.

The cartridge contained three substantially planar membranes positioned and spaced relative to each other to form two chambers through which sample or solvent can be passed. A separation membrane was positioned between two outer membranes (termed restriction membranes as their molecular mass cut-offs are usually smaller than the cut off of the separation membrane). When the cartridge was installed in the apparatus, the restriction membranes were located adjacent to an electrode. The cartridge is described in AU 738361, which description is incorporated herein by reference.

Description of membrane-based electrophoresis can be found in U.S. Pat. No. 5,039,386 and U.S. Pat. No. 5,650,055 in the name of Gradipore Limited, which description is incorporated herein by reference.

Polyacrylamide Gel Electrophoresis (PAGE)

Standard PAGE methods were employed as set out below.

Reagents: 10× SDS Glycine running buffer (Gradipore Limited, Australia), dilute using Milli-Q water to 1× for use; 1× SDS Glycine running buffer (29 g Trizma base, 144 g Glycine, 10 g SDS, make up in RO water to 1.0 l); 10× TBE II running buffer (Gradipore), dilute using Milli-Q water to 1× for use; 1× TBE II running buffer (10.8 g Trizma base, 5.5 g Boric acid, 0.75 g EDTA, make up in RO water to 1.0 l); 2× SDS sample buffer (4.0 ml, 10% (w/v) SDS electrophoresis grade, 2.0 ml Glycerol, 1.0 ml 0.1% (w/v) Bromophenol blue, 2.5 ml 0.5M Tris-HCl, pH 6.8, make up in RO water up to 10 ml); 2× Native sample buffer (10% (v/v) 10× TBE II, 20% (v/v) PEG 200, 0.1 g/l Xylene cyanole, 0.1 g/l Bromophenol blue, make up in RO water to 100%); Coomassie blue stain (Gradipure™, Gradipore Limited). Note: contains methanol 6% Acetic Acid solution for de-stain.

Molecular weight markers (Recommended to store at −20° C.): SDS PAGE (e.g. Sigma wide range); Western Blotting (e.g. color/rainbow markers).

SDS PAGE with non-reduced samples

To prepare the samples for running, 2× SDS sample buffer was added to sample at a 1:1 ratio (usually 50 μl/50 μl) in the microtiter plate wells or 1.5 ml tubes. The samples were incubated for 5 minutes at approximately 100° C. Gel cassettes were clipped onto the gel support with wells facing in, and placed in the tank. If only running one gel on a support, a blank cassette or plastic plate was clipped onto the other side of the support Sufficient 1× SDS glycine running buffer was poured into the inner tank of the gel support to cover the sample wells. The outer tank was filled to a level approximately midway up the gel cassette. Using a transfer pipette, the sample wells were rinsed with the running buffer to remove air bubbles and to displace any storage buffer and residual polyacrylamide.

Wells were loaded with a minimum of 5 μl of marker and the prepared samples (maximum of 40 μl). After placing the lid on the tank and connecting leads to the power supply the gel was run at 150V for 90 minutes. The gels were removed from the tank as soon as possible after the completion of running, before staining or using for another procedure (e.g. Western blot).

Staining and De-Staining of Gels

The gel cassette was opened to remove the gel which was placed into a container or sealable plastic bag. The gel was thoroughly rinsed with tap water, and drained from the container. Coomassie blue stain (approximately 100 ml Gradipure™, Gradipore Limited, Australia)) was added and the container or bag sealed. Major bands were visible in 10 minutes but for maximum intensity, stained overnight. To de-stain the gel, the stain was drained off from the container.

The container and gel were rinsed with tap water to remove residual stain. 6% acetic acid (approximately 100 ml) was poured into the container and sealed. The de-stain was left for as long as it takes to achieve the desired level of de-staining (usually 12 hours). Once at the desired level, the acetic acid was drained and the gel rinsed with tap water.

Size exclusion electrophoresis

Compared to column chromatography, which normally involve high pressure drops and compaction for soft gels at high flow rates, membrane chromatography has a lower pressure drop, high flow rate and high productivity as result of microporous/macroporous structures in relatively thin membranes.

As described above, protein separations under electrophoresis with a separation membrane are normally either size or charge based, which have limitations of its own such as the range of proteins can be separated. The present inventors have introduced the concept of protein or other compound separation under size exclusion chromatography principle using electrophoresis. By using this concept, protein or compound can be separated in an opposite manner to conventional electrophoresis and some large biomolecules, which are not able to be separated by existing systems, have been purified by this process.

The basic requirements for a SE separation are that the separation medium contains at least two types of pores: macropores and micropores. In chromatography, the large molecules will go though the big pores and travel fast while the smaller molecules will have interaction with small pores due to its compatible size with the micropores. Therefore in the separation of polymers by using size exclusion chromatography, polymer with largest molecular weight will elute out of a separating column first and the one with the smallest molecular weight will elute out last.

In the design of the SE hydrogel matrix systems, the present inventors have adopted the same principle. The solvent system used can act both as a porogen and a solvent to the amphiphilic monomer. The monomers used produce network structures with functional groups and these functional groups can interact with small proteins as these molecules enter the small pore structure.

The hydrogels can be used in two different ways by utilizing the recently developed Gradiflow™ system to test the separation of the resultant membranes; one way is for the manufacture of membranes with a larger pore size or with improved functionality. The other is SE hydrogel electrophoresis.

Membranes with larger pore size can be tested in the following way: the membrane will be placed in the middle of a separation cartridge in a separation unit. The protein mixture to be separated will be placed in stream 1. When the charge is applied, the separation will begin and small proteins will travel to downstream through the membranes.

When SE type membrane is used, it is placed in the middle of a separation cartridge in a separation unit. The protein mixture to be separated will be placed in stream 1. When the electric potential is applied, the separation will begin and large proteins will travel to downstream through the SE-type membranes. With the increase of time, small proteins may saturate the small pores of the separation membrane and the process needs to be pulsed to release the small proteins back to the upstream. This process can be carried out by removing separated proteins from downstream and reverse the potential supplied.

### DEFINITIONS

The following terms shall have the indicated definitions unless otherwise indicated:

“Hydrogel” is a chemically crosslinked polymer characterized by hydrophilicity and insolubility in water.

“Micropores” are pores within the gel network of the background matrix. The size of these pores can be related to the hydrogel formation species in the initial pre-gelling mixture using relationships and theories developed for common electrophoretic matrixes. For example, micropores within an acrylamide hydrogel are related to the total monomer concentration and monomer to crosslinker ratios in the free radical polymerization of acrylamide and N,N′-methylenebisacrylamide (Bansil, R.; Gupta, M. Ferroelectrics 1980, 30, 64).

“Macropores” are pores within the membrane that are significantly larger (more than 2 times) than micropores of the background matrix.

“Microporous membranen” is a separation membrane having substantially continuous interconnecting micropores. Such membranes are used extensively in preparative electrophoresis.

“Macroporous membrane” is a separation membrane having continuous interconnecting micropores but non-continuous macropores (i.e. macropores are not connected directly to each other). Such membranes have similar sieving properties to the corresponding microporous membrane, but allows for higher flow rate through the matrix because of the reduced diffusional constraints.

“Size exclusion membrane (SE-Mem)” is a bi, or multimodal separation membrane having continuous interconnecting micropores, and interconnecting macropores within its matrix. SE-Mem can have different separation behaviours depending upon the size of the micropores (S_{mic}), the size of the macropores (S_{mac}) and the size of the bio-molecule mixture (S_{bio}). When S_{bio}>S_{mac}>S_{mic}, no separation would occur, when S_{mac}˜S_{bio}>S_{mic}, all molecules with dimension smaller than the macropores would be separated from their bigger counter part, when S_{mac}>S_{bio}˜S_{mic}, all molecules with dimension smaller than the macropores would be separated from their bigger counter part, and be eluted in the order of decreasing size.

From the above description of SE-Mem, the challenge in producing such membrane lies in i) increase the size exclusion limit, i.e. the size of the largest interconnecting pores, and ii) produce a polymer with both interconnecting micropores and macropores. It would be a substantial advantage to develop a simple process to synthesis such membrane.

Multi-modal HEMA hydrogels are suitable to be used as SE-Mem as two general types of pores exist in such membrane - macropores formed by the template or porogen, and micropores formed by the crosslinking of polymer chains. The size exclusion limit of such membrane is also increased because of the macropores.

SE-Mem can be used in membrane based electrophoresis techniques and as membrane support for membrane chromatography and affinity membrane chromatography. It can take the form of flat sheet, stacked sheet, radical flow cartridges, hollow fibre molecules, slab, and column.

The term “stream 1 (S1)” refers to denote the first interstitial volume where sample is supplied in a stream to the electrophoresis apparatus. This stream may also be called the “upstream”.

The term “stream 2 (S2)” is used in this specification to denote the second interstitial volume where material is moved from the first interstitial volume through the separation membrane to a stream of the electrophoresis apparatus. This stream may also be called the “downstream”.

The term “forward polarity” is used when the first electrode is the cathode and the second electrode is the anode in the electrophoresis apparatus and current is applied accordingly.

The term “reverse polarity” is used when polarity of the electrodes is reversed such that the first electrode becomes the anode and the second electrode becomes the cathode.

### ABBREVIATIONS

Acrylamide (AAm); N,N′-methylenebisacrylamide (BIS); poly(acrylamide) gel electrophoresis (PAGE); 2-hydroxyethyl acrylate (HEA); 2-hydroxyethyl methacrylate (HEMA); poly(ethylene glycol) acrylate (PEGA); poly(ethylene glycol) methacrylate (PEGMA); ethylene glycol diacrylate (EGDA); ethylene glycol dimethacrylate (EGDMA); poly(ethylene glycol) acrylate (PEGA); poly(ethylene glycol) methacrylate (PEGMA); poly(ethylene glycol) diacrylate (PEGDA); poly(ethylene glycol) dimethacrylate (PEGDMA); poly(ethylene glycol) PEG; and poly(propylene glycol) PPG; poly(ethylene glycol) methyl ether PEGME; N,N,N′N′-tetramethylethylenediamine (TEMED); ammonium persulfate (APS).

### EXAMPLES

### Example 1

### Preparation of Monomer Solutions

Two terms are introduced to classify the monomer solutions:

%M refers to the total concentration of monomer as a weight percentage; %X refers to the number of double bonds on the crosslinkers as a portion of the total number of double bonds on the monomers.

$$\% M = \frac{total mass of monomers ( g )}{mass of reaction mixture ( g )} \times 100$$

$$\% X = \frac{number of double bonds on crosslinkers ( mol )}{total numbers of double bonds on monomers ( mol )} \times 100$$

Preparation of Acrylamide Hydrogels

### Example 2

### Preparation of 10%M 2%X AAm/BIS hydrogels for swelling tests using water as solvent

Monomer solutions (10 g) were prepared by dissolving AAm (978.3 mg) and BIS (21.7 mg) in water (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

### Example 3

### Preparation of 10%M 2%X AAm/BIS hydrogels for swelling tests using aqueous ethylene glycol as solvent

Aqueous solutions of ethylene glycol (25, 50 and 75%) were prepared by varying amounts of ethylene glycol and water. AAm (978.3 mg) and BIS (21.7 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

### Example 4

### Preparation of 10%M 2%X AAm/BIS hydrogels for swelling tests using aqueous propylene glycol as solvent

Aqueous solutions of propylene glycol (25, 50 and 75%) were prepared by varying amounts of ethylene glycol and water. AAm (978.3 mg) and BIS (21.7 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

### Example 5

### Preparation of 10%M 2%X AAm/BIS hydrogels for swelling tests using aqueous tri(ethylene glycol) as solvent

Aqueous solutions of triethylene glycol (22, 44, 67 and 72%) were prepared by varying amounts of triethylene glycol and water. AAm (978.3 mg) and BIS (21.7 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

### Example 6

### Preparation of 10%M 2%X AAm/BIS hydrogels for swelling tests using aqueous poly(ethylene glycol) 400 as solvent

Aqueous solutions of poly(ethylene glycol) 400 (6, 11, 16 and 22%) were prepared by varying amounts of poly(ethylene glycol) 400 and water. AAm (978.3 mg) and BIS (21.7 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

### Example 7

### Preparation of 10%M 2%X AAm/BIS hydrogels for turbidity measurements using aqueous tri(ethylene glycol) as solvent

Aqueous solutions of tri(ethylene glycol) (11, 22, 33, 44, 55, 61, 64, 66, 69 and 72%) were prepared by varying amounts of tri(ethylene glycol) and water. AAm (978.3 mg) and BIS (21.7 mg) was added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 8

### Preparation of 10%M 2%X AAm/BIS hydrogels for turbidity measurements using aqueous poly(ethylene glycol) 400 as solvent

Aqueous solutions of poly(ethylene glycol) 400 (6, 11, 16, 19, 22, 27 and 33%) were prepared by varying amounts of poly(ethylene glycol) 400 and water. AAm (978.3 mg) and BIS (21.7 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 9

### Preparation of 10%M 2%X AAm/BIS hydrogels for turbidity measurements using aqueous poly(ethylene glycol) 400 as solvent at 40° C.

Aqueous solutions of poly(ethylene glycol) 400 (6, 11, 16, 19, 22, 27 and 33%) were prepared by varying amounts of poly(ethylene glycol) 400 and water. AAm (978.3 mg) and BIS (21.7 mg) were added to the above solutions (9 g) in disposable glass vials. The monomer solution was then placed in a 40° C. water bath for 15 mins and degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at 40° C. for 2 hr under an argon environment.

### Example 10

### Preparation of 10%M 2%X AAm/BIS hydrogels for turbidity measurements using aqueous poly(ethylene glycol) 20,000 as solvent

Aqueous solutions of poly(ethylene glycol) 20,000 (0.02, 0.04, 0.06, 0.08, 0.1, 0.12 and 0.14%) were prepared by varying amounts of poly(ethylene glycol) 20,000 and water. AAm (978.3 mg) and BIS (21.7 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 11

### Preparation of 10%M 2%X AAm/BIS hydrogel cassettes for gel electrophoresis using water as solvent

10%M 2%X solutions (10 g) were prepared by dissolving AAm (978.3 mg) and BIS (21.7 mg) in water (6.5 g) and 1.5M Tris-HCl buffer (pH 8.8, 2.5 g). The stock buffer solution was prepared by dissolving Tris (27.23 g) in water (80 ml) and adjusted to the pH of 8.8 with 6 N HCl followed by making up the required volume (150 ml) with water.

The monomer solution was degassed by argon purging for 5 min prior to the addition of the initiator system (0.2 mol % initiator per double bond) composed of freshly made up 10% (w/v) APS (64.1 μm) and 10% (v/v) TEMED (42.4 μm). The gel solution (7 ml) was then immediately cast between two glass plates (8×8 cm, 1 mm apart) that were purged with argon and left to polymerize at room temperature for 3 hr under an argon environment prior to use.

### Example 12

### Preparation of 10%M 2%X AAm/BIS hydrogel cassettes for gel electrophoresis using 25% aqueous ethylene glycol as solvent

10%M 2%X solutions (10 g) were prepared by dissolving AAm (978.3 mg) and BIS (21.7 mg) in ethylene glycol (2.7 g) and water (3.8 g). 1.5M Tris-HCl buffer (pH 8.8, 2.5 g). The stock buffer solution was prepared by dissolving Tris (27.23 g) in water (80 ml) and adjusted to the pH of 8.8 with 6 N HCl followed by making up the required volume (150 ml) with water.

The monomer solution was degassed by argon purging for 5 min prior to the addition of the initiator system (0.2 mol % initiator per double bond) composed of freshly made up 10% (w/v) APS (64.1 μm) and 10% (v/v) TEMED (42.4 μm). The gel solution (7 ml) was then immediately cast between two glass plates (8×8 cm, 1 mm apart) that were purged with argon and left to polymerize at room temperature for 3 hr under an argon environment prior to use.

### Example 13

### Preparation of 10%M 2%X AAm/BIS hydrogel cassettes for gel electrophoresis using 25% aqueous propylene glycol as solvent

10%M 2%X solutions (10 g) were prepared by dissolving AAm (978.3 mg) and BIS (21.7 mg) in propylene glycol (2.7 g) and water (3.8 g). 1.5M Tris-HCl buffer (pH 8.8, 2.5 g). The stock buffer solution was prepared by dissolving Tris (27.23 g) in water (80 ml) and adjusted to the pH of 8.8 with 6 N HCl followed by making up the required volume (150 ml) with water.

The monomer solution was degassed by argon purging for 5 min prior to the addition of the initiator system (0.2 mol % initiator per double bond) composed of freshly made up 10% (w/v) APS (64.1 μm) and 10% (v/v) TEMED (42.4 μm). The gel solution (7 ml) was then immediately cast between two glass plates (8×8 cm, 1 mm apart) that were purged with argon and left to polymerize at room temperature for 3 hr under an argon environment prior to use.

### Example 14

### Preparation of 10M 2X AAm/BIS hydrogel cassettes for gel electrophoresis using 11% aqueous tri(ethylene glycol) as solvent

10%M 2%X solutions (10 g) were prepared by dissolving AAm (978.3 mg) and BIS (21.7 mg) in tri(ethylene glycol) (1.2 g) and water (5.3 g). 1.5M Tris-HCl buffer (pH 8.8, 2.5 g). The stock buffer solution was prepared by dissolving Tris (27.23 g) in water (80 ml) and adjusted to the pH of 8.8 with 6 N HCl followed by making up the required volume (150 ml) with water.

The monomer solution was degassed by argon purging for 5 min prior to the addition of the initiator system (0.2 mol % initiator, per double bond) composed of freshly made up 10% (w/v) APS (64.1 μm) and 10% (v/v) TEMED (42.4 μm). The gel solution (7 ml) was then immediately caste between two glass plates (8×8 cm, 1 mm apart) that were purged with argon and left to polymerize at room temperature for 3 hr under an argon environment prior to use.

### Example 15

### Preparation of 10%M 2%X AAm/BIS hydrogel cassettes for gel electrophoresis using 5.5 and 11% aqueous poly(ethylene glycol) 400 as solvent

10%M 2%X solutions (10 g) were prepared by dissolving AAm (978.3 mg) and BIS (21.7 mg) in poly(ethylene glycol) 400 (0.6 or 1.2 g) and water (5.3 or 5.9 g). 1.5M Tris-HCl buffer (pH 8.8, 2.5 g). The stock buffer solution was prepared by dissolving Tris (27.23 g) in water (80 ml) and adjusted to the pH of 8.8 with 6 N HCl followed by making up the required volume (150 ml) with water.

The monomer solution was degassed by argon purging for 5 min prior to the addition of the initiator system (0.2 mol % initiator per double bond) composed of freshly made up 10% (w/v) APS (64.1 μm) and 10% (v/v) TEMED (42.4 μm). The gel solution (7 ml) was then immediately cast between two glass plates (8×8 cm, 1 mm apart) that were purged with argon and left to polymerize at room temperature for 3 hr under an argon environment prior to use.

Evaluation of Acrylamide Hydrogels

Swelling Tests

Gels made according to Examples 2-6 were immersed in water (500 g) for 1 week during which the immersing solution (water) was exchanged on a daily basis. The gel was then dried in a 40° C. oven for 1 week. The equilibrium solvent content of the gel was determined by the following equation.

$$Equilibrium solvent content ( ESC ) = \frac{weight ( swollen gel ) - weight ( dried gel )}{weight ( dried gel )}$$

### Example 16

### ESC (Water) of AAm/BIS hydrogels synthesized in water and aqueous solutions of ethylene glycol

| Polymerization Solvent | ESC |
|---|---|
| water | 12.1 |
| 25% ethylene glycol/75% water | 14.3 |
| 50% ethytene glycol/50% water | 15.4 |
| 75% ethylene glycol/25% water | 20.0 |

### Example 17

### ESC (water) of AAm/BIS hydrogels synthesized in water and aqueous solutions of propylene glycol

| Polymerization Solvent | ESC |
|---|---|
| water | 12.1 |
| 25% propylene glycol/75% water | 15.3 |
| 50% propylene glycol/50% water | 21.9 |
| 75% propylene glycol/25% water | 28.6 |

### Example 17

### ESC (water) of AAm/BIS hydrogels synthesized in water and aqueous solutions of tri(ethylene glycol)

| Polymerization Solvent | ESC |
|---|---|
| water | 12.1 |
| 11% tri(ethylene glycol)/89% water | 12.7 |
| 22% tri(ethylene glycol)/78% water | 14.5 |
| 33% tri(ethylene glycol)/66% water | 16.3 |
| 44% tri(ethylene glycol)/56% water | 18.1 |
| 55% tri(ethylene glycol)/45% water | 21.8 |
| 61% tri(ethylene glycol)/39% water | 25.0 |
| 64% tri(ethylene glycol)/36% water | 26.3 |
| 66% tri(ethylene glycol)/34% water | 26.7 |
| 69% tri(ethylene glycol)/31% water | 30.0 |
| 72% tri(ethylene glycol)/28% water | 32.8 |

### Example 17

### ESC (water) of AAm/BIS hydrogels synthesized in water and aqueous solutions of poly(ethylene glycol) 400

| Polymerization Solvent | ESC |
|---|---|
| water | 12.1 |
| 6% poly(ethylene glycol) 400/94% water | 13.2 |
| 11% poly(ethylene glycol) 400/89% water | 14.0 |
| 16% poly(ethylene glycol) 400/84% water | 15.2 |
| 22% poly(ethylene glycol) 400/78% water | 17.3 |

Turbidity Measurements

The turbidity of gels made according to examples 7-9 was measured using UV-visible spectrophotometry. Distilled water was used for the baseline and the absorbance of each gel sample and the corresponding polymerization solvent were recorded at 100 nm intervals between 300 and 800 nm. The turbidity of the gel samples were determined by the following equation.
Turbidity=−logₑ(10^{-(absorbance of gel-absorbance of polymerization solvent)})

### Example 18

### Turbidity of 10%M 2%X AAm/BIS hydrogels synthesized in water and aqueous solutions of poly(ethylene glycol) 400 at 500 nm (room temperature and 40° C.)

| | Turbidity | |
|---|---|---|
| | (Room | Turbidity |
| Polymerization Solvent | Temperature) | (40° C.) |
| Water | 0 | 0 |
| 6% poly(ethylene glycol) 400/94% water | 0 | 0 |
| 11% poly(ethylene glycol) 400/89% water | 0 | 0 |
| 16% poly(ethylene glycol) 400/84% water | 0.23 | 0 |
| 19% poly(ethylene glycol) 400/81% water | 0.46 | 0.18 |
| 22% poly(ethylene glycol) 400/78% water | 1.27 | 0.32 |
| 27% poly(ethylene glycol) 400/73% water | 6.90 | 5.4 |
| 33% poly(ethylene glycol) 400/66% water | 8.06 | 7.5 |

*Visual opacity corresponds to a turbidity value of >0.3 at 500 nm

### Example 19

### Turbidity of 10%M 2% AAm/BIS hydrogels synthesized in water and aqueous solutions of tri(ethylene glycol), poly(ethylene glycol) 400 and poly(ethylene glycol 20,000 at 500 nm (room temperature)

Turbidity testing showed that the onset of opacity occurs at 72%, 19% and 0.1% for aqueous solution of tri(ethylene glycol), poly(ethylene glycol) 400 and poly(ethylene glycol) 20,000 respectively.

Gel Electrophoresis

Standard SDS-PAGE was performed on the acrylamide hydrogel cassette (example 11-15) using a constant voltage of 150 V and Tris-glycine electrophoresis running buffer. The electrophoresis running buffer (100 ml) was prepared by dissolving Tris (9 g), SDS (3 g), and glycine (43.2 g) in water and diluting 1:5 with water before use. 10 μL of Kaleidoscope pre-stained protein marker or SDS-PAGE molecular weight standards (broad range) was syringed into sample wells and separated. Gels with SDS-PAGE molecular weight standards (broad range) were stained for 3 hr using Coomassie Blue solution and de-stained overnight with 10% aqueous acetic acid. The migration ratio of a protein was determined by the following equation.

$$Migration Ratio = \frac{distance travelled by protein}{disctance travelled by dyefront}$$

Kaleidoscope Prestained Standards (Bio-Rad 161-0324)

| Protein | Calibrated MW |
|---|---|
| Myosin | 206,000 |
| β-galactosidase | 128,000 |
| Bovine serum albumin | 81,000 |
| Carbonic anhydrase | 40,300 |
| Soybean trypsin inhibitor | 31,600 |
| Lysozyme | 19,300 |
| Aprotinin | 7,800 |

SDS-PAGE Molecular Weight Standards
(board range, Bio-Rad 161-0317)

| Protein | Calibrated MW |
|---|---|
| Myosin | 200,000 |
| β-galactosidase | 116,250 |
| Phosphorylase b | 97,400 |
| Serum albumin | 66,200 |
| Ovalbumin | 45,000 |
| Carbonic anhydrase | 31,000 |
| Trypsin inhibitor | 21,500 |
| Lysozyme | 14,400 |
| Aprotinin | 6,500 |

### Example 20

### Electrophoresis of 10%M 2%X AAm/BIS gel cassette synthesized in water and aqueous solutions of ethylene glycol or propylene glycol

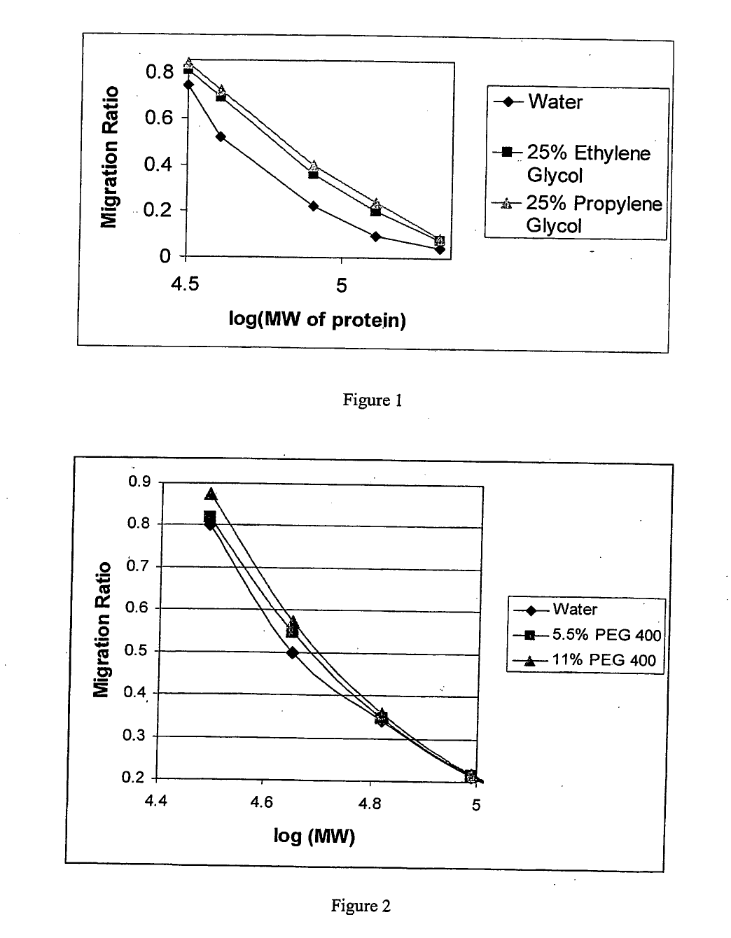

Migration ratios of Kaleidoscope Pre-stained Standards in 10%M 2%X acrylamide gel cassette synthesized in water, aqueous solutions of ethylene glycol (25%) or propylene glycol (25%) are shown in FIG. 1.

### Example 21

### Electrophoresis of 10%M 2%X acrylamide gel cassette synthesized in water and aqueous solutions of poly(ethylene glycol) 400

Migration ratios of SDS-PAGE Molecular Weight Standards (board range) in 10%M 2%X acrylamide gel cassette synthesized in water, or aqueous solutions of poly(ethylene glycol) are shown in FIG. 2.

### Example 22

### Electrophoresis of 10%M 2%X AAm/BIS gel cassette synthesized in water and aqueous solutions of tri(ethylene glycol) or poly(ethylene glycol) 400

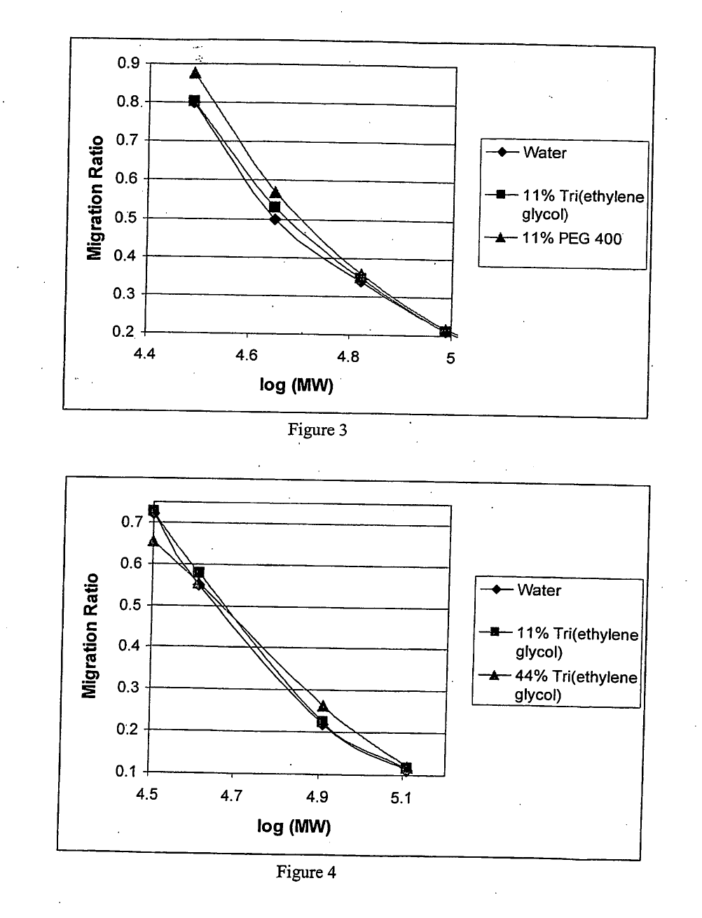

Migration ratios of SDS-PAGE Molecular Weight Standards (board range) in 10%M 2%X acrylamide gel cassette synthesized in water or aqueous solutions of tri(ethylene glycol) and poly(ethylene glycol) are shown in FIG. 3.

### Example 23

### Electrophoresis of 10%M 2%X AAm/BIS gel cassette synthesized in water and aqueous solutions of tri(ethylene glycol)

Migration ratios of Kaleidoscope Prestained Standards in 10%M 2%X acrylamide gel cassette synthesized in water and aqueous solutions of tri(ethylene glycol) are shown in FIG. 4.

Preparation of Methacrylamide Hydrogels

### Example 24

### Preparation of 10%M 2%X methacrylamide/N,N′-methylenebismethacrylamide hydrogels using aqueous glycerol as solvent

Aqueous solution of glycerol (75%) were prepared by mixing appropriate amount of water and glycerol methacrylamide (978.6 mg) and N,N′-methylenebismethacrylamide (21.4 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment to produce a hydrogel that was visually clear.

Opacity and reduction in mechanical integrity was observed when the above methacrylamide hydrogel was equilibrated in water.

Preparation of 2-Hydroxyethyl Acrylate (HEA) Hydrogels

### Example 25

### Preparation of HEA/EGDA hydrogels using water as solvent

10%M HEA hydrogels at 3, 4, 5, 6, and 10%X were prepared by mixing the appropriate amount of HEA, EGDA. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

All of the resultant polymers were not visually clear, the opacity was observed to increase with increasing %X.

### Example 26

### Preparation of 10%M 6.5%X HEA/EGDA hydrogels using aqueous ethylene glycol as solvent

Aqueous solutions of ethylene glycol (20, 40, 60 and 80%) were prepared by varying amounts of ethylene glycol and water. HEA (951.5 mg) and EGDA (48.5 mg) were added to the above solutions (9 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

The polymers synthesized in 0 and 20% ethylene glycol solutions were opaque. The polymer synthesized in 40% ethylene glycol solution was slightly opalescence. The polymer synthesized in 60 and 80% ethylene glycol solutions were visually clear and remained visually clear after equilibration in water.

### Example 27

### Preparation of 10%M 6.5%X HEA/EGDA hydrogels using aqueous solutions of poly(ethylene glycol) 200, tetrahydrofuran, or methanol as solvent

60% aqueous solutions of PEG 200, tetrahydrofuran, or methanol were prepared. HEA (951.5 mg) and EGDA (48.5 mg) were added to the above solutions (9 g) in disposable glass vials. The monomer solution was then degassed by argon purging for 5 min prior to the addition of the initiator system (0.2 mol % initiator per double bond) composed of freshly made up 10%.(w/v) APS and 10% (v/v) TEMED. The polymerization was then allowed to proceed at room temperature overnight under an argon environment.

The polymers synthesized in 60% PEG 200, 60% tetrahydrofuran, and 60% methanol were visually clear. All gels were visually clear and remained visually clear after equilibration in water.

Preparation of 2-Hydroxyethyl Methacrylate (HEMA) Hydrogels

### Example 28

### Preparation of 5%X HEMA/EGDMA hydrogels using water as solvent

10%, 20%, 30% and 40%M HEMA hydrogels were prepared by mixing the appropriate amount of HEMA, EGDMA and water (10 g total) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

All of the resultant polymers were highly opaque and had little mechanical strength.

### Example 29

### Preparation of 15%M 5%X HEMA/EGDMA hydrogels for turbidity measurements using aqueous ethylene glycol, tri(ethylene glycol), PEG 400 or PEG 6,000 as solvent

Aqueous solutions of ethylene glycol, tri(ethylene glycol), PEG 400 or PEG 6,000 (40, 45, 50, 60 and 70%) were prepared. HEMA (1.442 g) and EGDMA (57.8 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 30

### Preparation of 15%M HEMA/EGDMA hydrogels for turbidity measurements using 50% PEG 200 as solvent

Aqueous solution of PEG 200 (50%) was prepared by mixing the appropriate amount of PEG 200 and water. 15%M HEMA hydrogels with 0, 2.5, 5, 7.5 and 10%X were prepared by mixing the appropriate amounts of HEMA, EGDMA and 50% PEG solution (10 g total) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 31

### Preparation of 5%X HEMA/EGDMA hydrogels for turbidity measurements using 50% PEG 200 as solvent

Aqueous solution of PEG 200 (50%) was prepared by mixing the appropriate amount of PEG 200 and water. 5%X HEMA hydrogels with 7.5, 10, 12.5, 15, 20, 40%T were prepared by mixing the appropriate amounts of HEMA, EGDMA and 50% PEC solution (10 g total) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 32

### Preparation of 15%M 5%X HEMA/EGDMA hydrogels for turbidity measurements using aqueous propylene glycol, tri(propylene glycol) or PPG 425 as solvent

Aqueous solutions of propylene glycol, tri(propylene glycol) or PPG 425 (30, 35, 40, 45 and 50%) were prepared. HEMA (1.442 g) and EGDMA (57.8 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 33

### Preparation of 15%M 5%X HEMA/EGDMA hydrogels for turbidity measurements using aqueous PEG dimethyl ether 500 as solvent

Aqueous solutions of PEG dimethyl ether 500 (30, 35, 40, 45 and 50%) were prepared. HEMA (1.442 g) and EGDMA(57.8 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 34

### Preparation of 15%M 5%X HEMA/EGDMA hydrogels for turbidity measurements using aqueous ethylene glycol monomethyl ether, ethylene glycol monoethyl ether or ethylene glycol monobutyl ether as solvent

Aqueous solutions of ethylene glycol monomethyl ether, ethylene glycol monoethyl ether or ethylene glycol monobutyl ether (30, 35, 40, 45 and 50%) were prepared. HEMA (1.442 g) and EGDMA (57.8 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 35

### Preparation of 15%M 5%X HEMA/EGDMA hydrogels for turbidity measurements using aqueous poly(ethylene glycol-co-propylene glycol) 2,500 (poly(eg-co-pg) 2,000), poly(ethylene glycol-co-propylene glycol) 12,000 (poly(eg-co-pg) 12,000) or poly(ethylene glycol-block-propylene glycol-block-ethylene glycol) 1,900 (poly(eg-b-pg-eg) 1,900) as solvent

Aqueous solutions of poly(eg-co-pg) 2,000, poly(eg-co-pg) 12,000 or poly(eg-b-pg-eg) 1,900 (30, 35, 40, 45 and 50%) were prepared. HEMA (1.442 g) and EGDMA (57.8 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 36

### Preparation of 15%M 5%X HEMA/EGDMA hydrogels for turbidity measurements using aqueous PEG 400 or PPG 425 as solvent

Aqueous solutions of PEG 400 or PPG 425 (30, 50, 70 and 90%) were prepared. HEMA (1.442 g) and EGDMA (57.8 mg) were added to the above solutions (8.59) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 37

### Preparation of 15%M 5%X HEMA/EGDMA hydrogels for turbidity measurements using aqueous solutions of poly(eg-b-pg-b-eg) 1900 and PEG 400 mixtures as solvent

40% aqueous solutions of poly(eg-b-pg-b-eg) 1900 and PEG 400 mixtures (0, 12.5, 25, 50, 75, 87.5 and 100% poly(eg-b-pg-b-eg) 1900) were prepared. HEMA (1.442 g) and EGDMA (57.8 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 38

### Preparation of 15%M 5%X HEMA/EGDMA hydrogels for turbidity measurements using aqueous solutions of ethylene glycol monomethyl ether and PEG 200 mixtures as solvent

35% aqueous solutions of ethylene glycol monomethyl ether and PEG 200 mixtures (0, 14, 28, 57, 86 and 100% ethylene glycol monomethyl ether) were prepared. HEMA (1.442 g) and EGDMA (57.8 mg) were added to the above solutions (8.5 g) in disposable glass vials. The monomer solution was, then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 39

### Preparation of 5%X HEMA/EGDMA hydrogels for swelling tests using aqueous tri(ethylene glycol) as solvent

Aqueous solution of tri(ethylene glycol) (60%) were prepared. 20, 40, 60 and 80%M HEMA/EGDMA hydrogels were prepared by mixing the appropriate amount of HEMA, EGDMA and the above 60% tri(ethylene glycol) solution in disposable glass vials (10 g total). The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

### Example 40

### Preparation of 10%M 5%X HEMA/EGDMA hydrogels for swelling tests using water and aqueous solutions of PEG 200 or PEG 4000 as solvent

Aqueous solutions of PEG 200 or PEG 4000 (50%) were prepared. HEMA (1.442 g) and EGDMA (57.8 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. The polymerization was then allowed to proceed at room temperature overnight under an argon environment.

### Example 41

### Preparation of 15%M 4%X HEMA/EGDMA membrane for electrophoretic separation analysis using aqueous solutions of PEG 200 as solvent

Unwoven poly(ethyleneterephthalate) (PET) sheets that served as a mechanical support were treated with aqueous solution of Teric BL8 (0.5% (v/v)), Huntsman Corp. Australia) a non-ionic surfactant used to improve surface wettability.

Aqueous solution of PEG 200 (80%) were prepared. 15%M 4%X HEMA/EGDMA mixtures with the above PEG 200 solution were polymerized into thin membranes with Teric BL8 treated unwoven PET sheet as the supporting substrate.

Evaluation of HEMA Hydrogels

Turbidity testing

All HEMA hydrogels which were visually clear after the synthesis remained visually clear after the solvent was exchanged with water.

### Example 42

### Turbidity of 15%M 5%X HEMA/EGDMA hydrogels synthesized in aqueous ethylene glycol, tri(ethylene glycol), PEG 400 or PEG 6,000 solutions at 500 nm

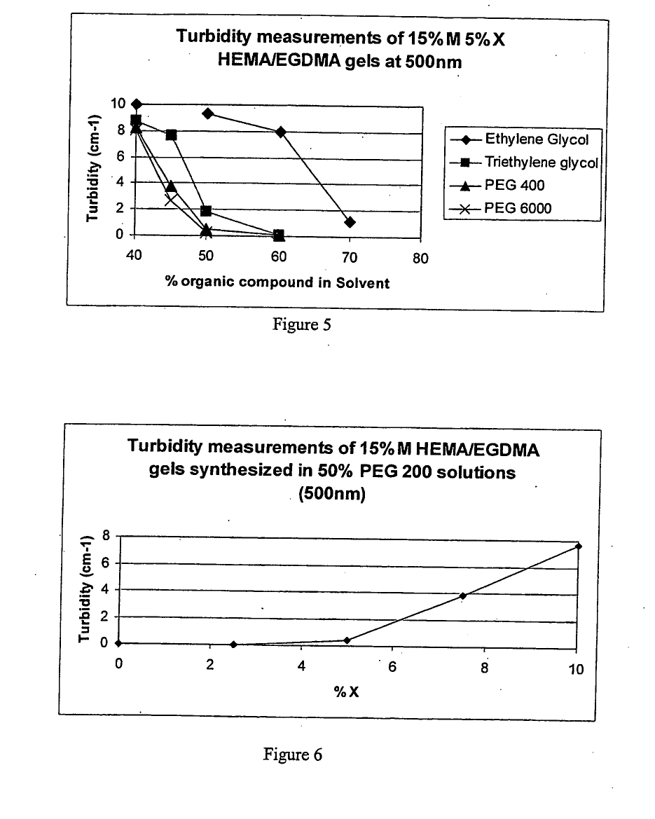

Turbidity results of polymers synthesized according to Example 29 are shown in FIG. 5.

### Example 43

### Turbidity of 15%M HEMA/EGDMA hydrogels synthesized in 50% aqueous PEG 200 solution at 500 nm

Turbidity results of polymers synthesized according to Example 30 are shown in FIG. 6.

### Example 44

### Turbidity of 5%X HEMA/EGDMA hydrogels synthesized in 50% aqueous PEG 200 solution at 500 nm

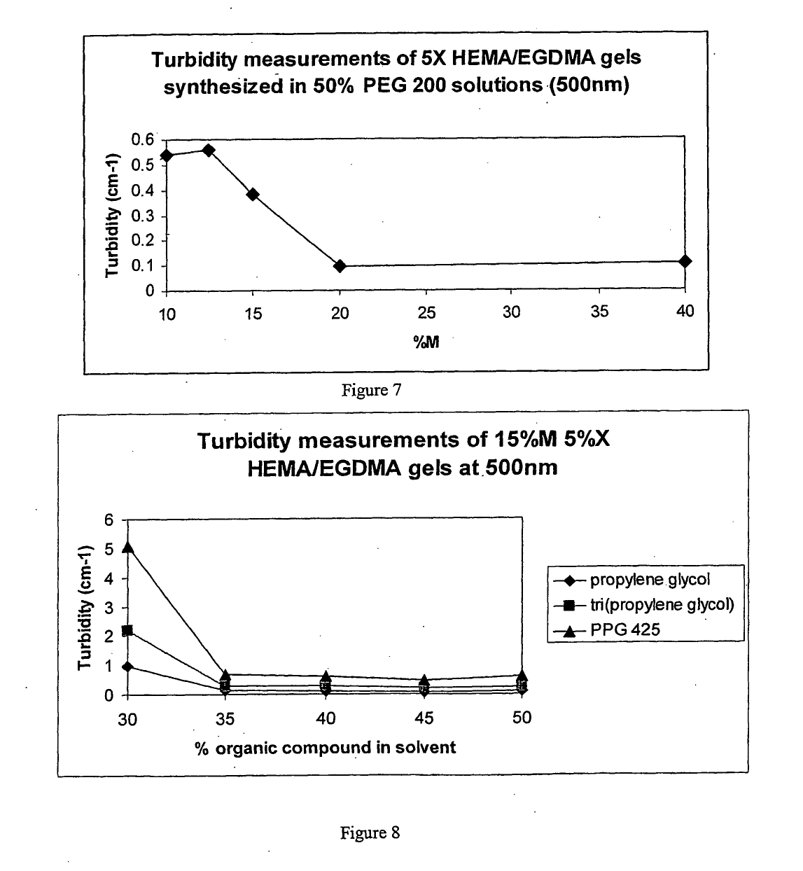

Turbidity results of polymers synthesized according to Example 31 are shown in FIG. 7.

### Example 45

### Turbidity of 15%M 5%X HEMA/EGDMA hydrogels synthesized in aqueous propylene glycol, tri(propylene glycol) or PPG 425 as solvent

Turbidity results of polymers synthesized according to Example 32 are shown in FIG. 8.

### Example 46

### Turbidity of 15%M 5%X HEMA/EGDMA hydrogels synthesized in aqueous PEG dimethyl ether 500 solutions

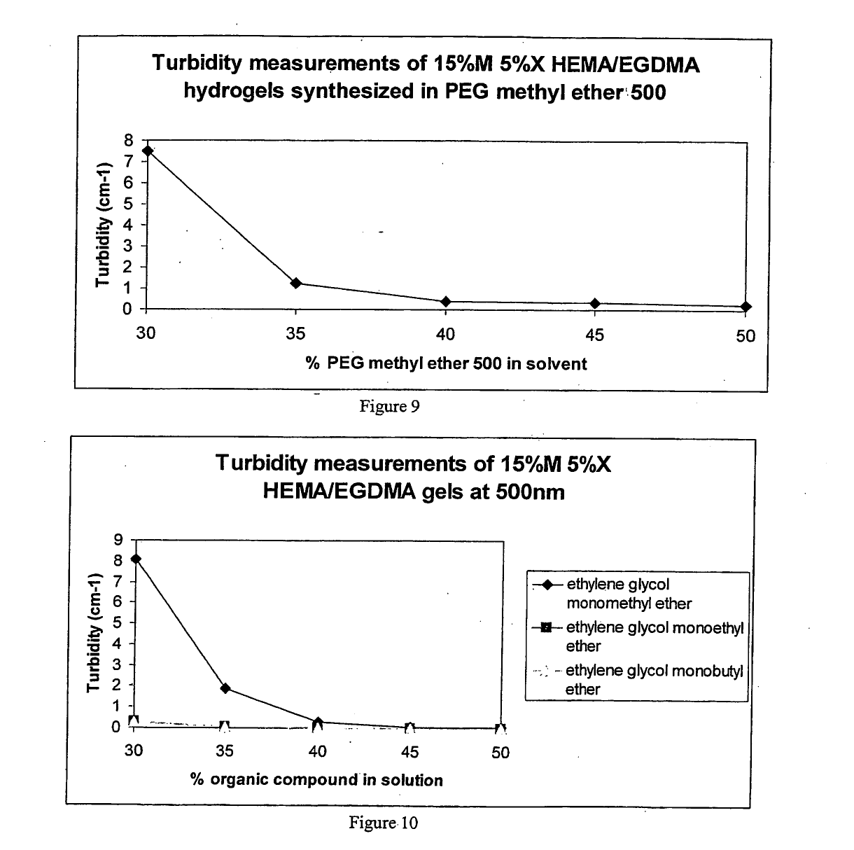

Turbidity results of polymers synthesized according to Example 33 are shown in FIG. 9.

### Example 47

### Turbidity of 15%M 5%X HEMA/EGDMA hydrogels synthesized in aqueous ethylene glycol monomethyl ether, ethylene glycol monoethyl ether or ethylene glycol monobutyl ether as solvent

Turbidity results of polymers synthesized according to Example 34 are shown in FIG. 10.

### Example 48

### Turbidity of 15%M 5%X HEMA/EGDMA hydrogels synthesized in aqueous poly(ethylene glycol-co-propylene glycol) 2,500 (poly(eg-co-pg) 2,000), poly(ethylene glycol-co-propylene glycol) 12,000 (poly(eg-co-pg) 12,000) or poly(ethylene glycol-block-propylene glycol-block-ethylene glycol) 1,900 (poly(eg-b-pg-eg) 1,900) as solvent

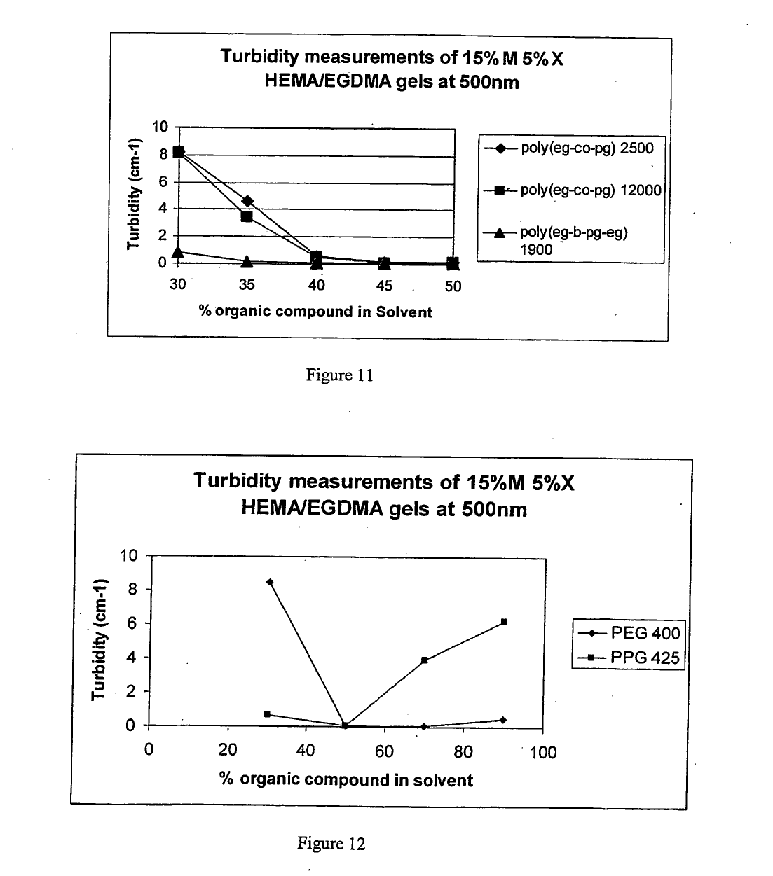

Turbidity results of polymers synthesized according to Example 35 are shown in FIG. 11.

### Example 49

### Turbidity of 15%M 5%X HEMA/EGDMA hydrogels synthesized in aqueous PEG 400 or PPG 425 as solvent

Turbidity results of polymers synthesized according to Example 36 are shown in FIG. 12.

### Example 50

### Turbidity of 15%M 5%X HEMA/EGDMA hydrogels synthesized in aqueous solutions of poly(eg-b-pg-b-eg) 1900 and PEG 400 mixtures as solvent

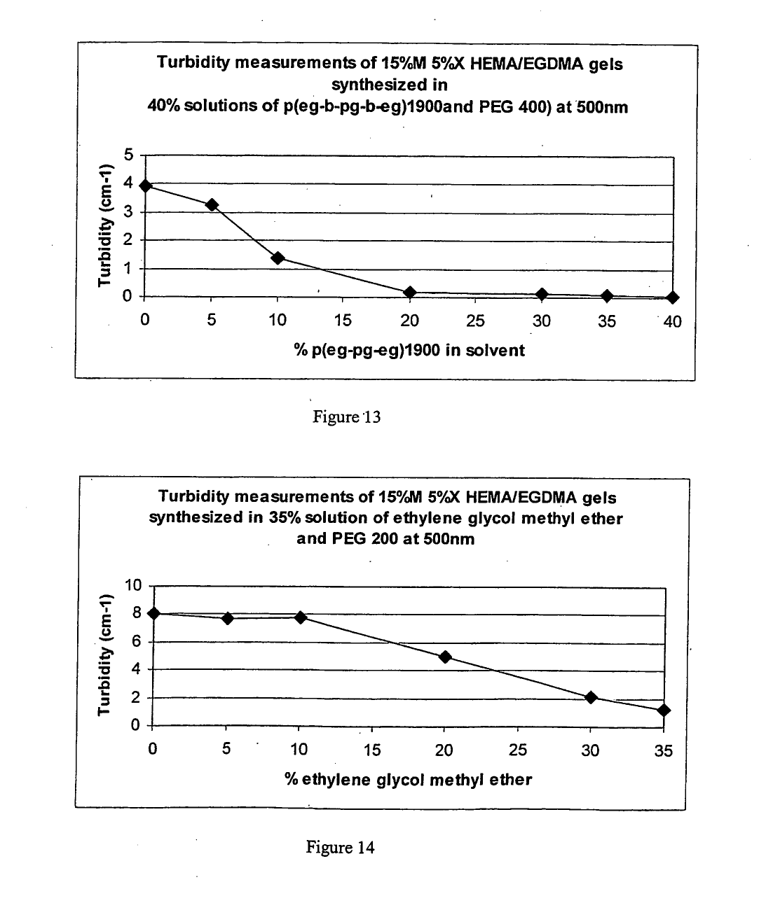

Turbidity results of polymers synthesized according to example 37 are shown in FIG. 13.

### Example 51

### Turbidity of 15%M 5%X HEMA/EGDMA hydrogels synthesized in aqueous solutions of ethylene glycol monomethyl ether and PEG 200 mixtures as solvent

Turbidity results of polymers synthesized according to Example 38 are shown in FIG. 14.

### Example 52

### Swelling test (water) of 5%X HEMA/EGDMA hydrogels at 20, 40, 60, 80%M synthesized in 60% aqueous tri(ethylene glycol) solution

| Hydrogel | ESC (water) |
|---|---|
| 20% M 5% X | 0.81 |
| 40% M 5% X | 0.72 |
| 60% M 5% X | 0.56 |
| 80% M 5% X | 0.54 |

### Example 53

### Swelling test (water) of 15%M 5%X HEMA/EGDMA hydrogels synthesized in 50% aqueous solutions of PEG 200 or PEG 4000

ESC(water) for 15%M 5%X HEMA/EGDMA hydrogel synthesized in 50% PEG 200 solution was found to be 0.65.

ESC(water) for 15%M 5%X HEMA/EGDMA hydrogel synthesized in 50% PEG 4000 solution was found to be 0.83.

### Example 54

### Swelling test (40% aqueous solutions of ethylene glycol, PEG 600, PEG 4000 or PEG 6000) of 15%M 5%X HEMA/EGDMA hydrogels synthesized in 50% aqueous solutions of PEG 200 or PEG 400

Hydrogels prepared in Example 40 were immersed in water (500 g) for 1 week during which the immersing solution (water) was exchanged on a daily basis. The gel was then dried in a 40° C. oven for 1 week. The dried gels were then immersed in 50% aqueous solutions of ethylene glycol, PEG 600, PEG 4000 or PEG 6000) for 1 week during which the immersing solution was exchanged on a daily basis. The ESC of the gels are shown in the following table.

| | | ESC | ESC | ESC |
|---|---|---|---|---|
| | ESC | (40% PEG | (40% PEG | (40% PEG |
| | (40% EG) | 600) | 4000) | 6000) |
| 15% M 5% X hydrogels | 0.98 | 2.99 | 1.31 | 1.14 |
| synthesized in 50% | | | | |
| PEG 200 | | | | |
| 15% M 5% X hydrogels | 1.30 | 3.48 | 3.00 | 2.45 |
| synthesized in 50% | | | | |
| PEG 4000 | | | | |

### Example 55

### Electrophoresis separation analysis of 15%M 4%X HEMA/EGDMA membrane synthesized in 80% aqueous PEG 200 solution

Samples of known molecular weight and size were run through a Gradiflow™ BF 200 unit to investigate the relative pore size formed in HEMA hydrogel networks. The protein standards were placed in a buffer solution and run by current from the stream 1 section of the unit above the membrane. Proteins smaller than the pores of the membrane will pass through the membrane into the stream 2 section of the unit. The larger proteins will be recycled back into the stream 1 section. Ten μl samples from both the two streams of the unit are taken every 10 minutes and detected using SDS-PAGE. The migration pattern should indicate what sized samples passed through the membrane. More details on the construction and operation of this unit can be found in U.S. Pat. No. 5,650,055, U.S. Pat. No. 5,039,386, WO 00/56792 and WO 00/13776, incorporated herein by reference.

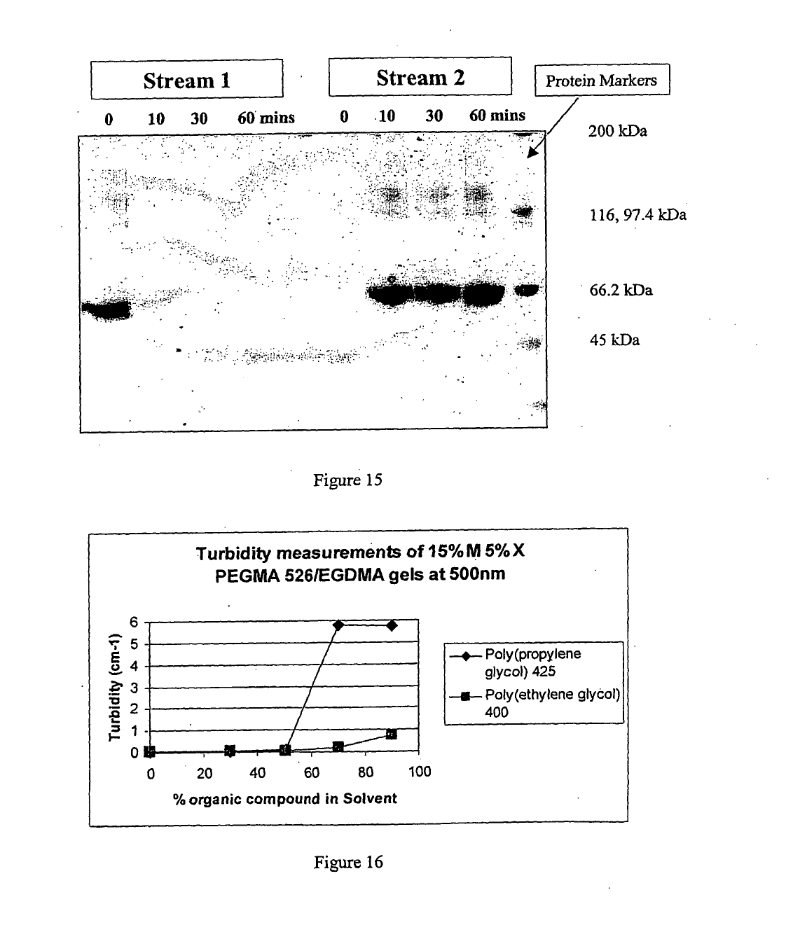

The separation and migration pattern of Bovine serum albumin (MW 67,000) by a 15%M 4%X HEMA/EGDMA membrane synthesized in 80% aqueous PEG 200 solution (Example 41) using 40 mM MES bis-TRIS buffer is shown in FIG. 15;

Preparation of Poly(ethylene glycol) Methacrylate (HEMA) Hydrogels

### Example 56

### Preparation of 15%M 5%X PEGMA 526/EGDMA hydrogels for turbidity measurements using aqueous PEG 400 or PPG 425 as solvent

Aqueous solutions of PEG 400 or PPG 425 (0, 30, 50 and 70%) were prepared. PEGMA (1.485 g) and EGDMA (14.7 mg) were added to the above solutions (8.5 g) in disposable glass vials. The monomer solution was then degassed by argon purging for 5 min prior to the addition of the initiator system (0.2 mol % initiator per double bond) composed of freshly made up 10% (w/v) APS and 10% (v/v) TEMED. Two 375 μl samples were pipetted into disposable cuvettes (10×10×45 mm³) and the polymerization was then allowed to proceed at room temperature overnight under an argon environment.

### Example 57

### Turbidity of 15%M 5%X PEGMA 526/EGDMA hydrogels synthesized in aqueous PEG 400 or PPG 425 as solvent

Turbidity results of polymers synthesized according to Example 56 are shown in FIG. 16.

Preparation of optically clear hydrogels

### Example 58

### ¹³C NMR relaxation measurements of acrylamide hydrogels

Monomer solutions (2 g) were prepared by dissolving AAm and Bis in the appropriate amount of D₂O (10% TMSPA-Na, 0.2 g), water and PEG-400. The monomer solution was then degassed by argon purging prior to the addition of the initiator system composed of freshly made up 10% (w/v) APS and 10% (v/v) TEMED (0.05 mol % initiator per double bond). This mixture was immediately pipetted into 5 mm NMR tube (0.38 mm wall thickness) and the polymerization was allowed to proceed at room temperature overnight under an argon environment.

¹³C NMR spectra were obtained using a Varian Unity Plus 400 spectrometer operating at 100 MHz. Spin-lattice relaxation times (T₁) were measured by the inversion-recovery method at 25° C. Recycled delays were set to 7s (>3T₁), with delay times (τ) of 10, 50, 100, 200, 300, 400, 500, 600, 700, 800, and 1000 ms. The T₁ parameters were calculated by fitting the data to the following equation.
l(τ)=l(τ=0) (1−2 exp(−τ/T₁)) (4)
when l is the intensity of the transformed peaks.

### Example 59

### Real-time viscosity measurements of acrylamide polymerizations

Monomer solutions (200 g) were prepared by dissolving AAm and Bis in the appropriate amount of water and PEG-400. The monomer solution was then degassed by argon purging prior to the addition of the initiator system composed of freshly made up 10% (w/v) APS and 10% (v/v) TEMED (0.05 mol % initiator per double bond). The viscosity of the reaction mixture was measured by a Brookfield® DV-II+ viscometer (0.3 rpm, LV-3 spindle). The experiments were performed in a glove box with controlled oxygen levels (<0.1% O₂).

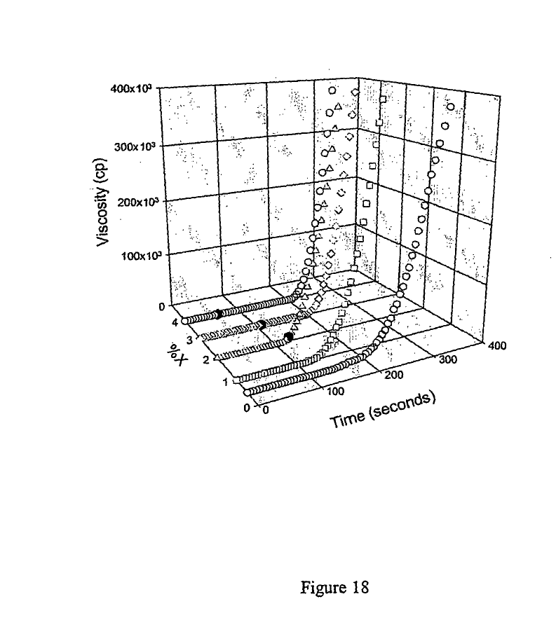

Viscosity measurements of the polymerizations are shown in FIG. 18. Times at which phase separation was observed in the samples are represented by dark coloured points (circle).

### Example 60

### Preparation of acrylamide hydrogels for swelling studies

Monomer solution (10 g) was prepared by dissolving AAm and Bis in an appropriate amount of water and PEG-400 in disposable glass vials. The monomer solution was then degassed by argon purging prior to the addition of the initiator system (0.2 mol % initiator per double bond), composed of freshly made up 10% (w/v) APS and 10% (v/v) TEMED. The polymerization was then allowed to proceed at room temperature overnight under an argon environment.

### Example 61

### Kinetic swelling studies of acrylamide hydrogels

The gel made according to the above procedure was immersed in water (500 g) for 1 week during which the immersing solution (water) was exchanged on a daily basis. The gel was then dried in a 40° C. oven for 1 week and re-swelled in water. The weight of the swollen gel was continuously monitored for 48 hours.
ESC of the gel was determined by the following equation:

$$Equilibrium solvent content ( ESC ) = \frac{weight ( swollen gel ) - weight ( dried gel )}{weight ( dried gel )}$$

### Example 62

### Preparation of 15%M 5%X HEMA/EGDMA hydrogels using aqueous ethylene glycol monomethyl ether as solvent

Aqueous solutions of ethylene glycol monomethyl ether (80, 85 and 90%) were prepared. HEMA (1.442 g) and EGDMA (57.8 mg) were added to the above solutions (8.5 g) in disposable glass vials. The monomer solution was then degassed by argon purging prior to the addition of the initiator system (0.2 mol % initiator per double bond) composed of freshly made up 10% (w/v) APS and 10% (v/v) TEMED. The polymerization was then allowed to proceed at room temperature overnight under an argon environment. All resultant gels were visually clear.

### Example 63

### ¹³C T₁ (25° C., 100 MHz) for 20%M 2%X acrylamide hydrogels synthesized in the presene of various amount of PEG-400

| % PEG-400 | T₁ (α-carbon) | T₁ (β-carbon) | T₁ (carbonyl) |
|---|---|---|---|
| 2.5 | 240 | 125 | 1330 |
| 7.5 | 240 | 135 | 1350 |
| 12.5 | 261 | 140 | 1400 |
| 17.5 | 270 | 155 | 1400 |
| 22.5 | 340 | 180 | 1730 |
| 27.5 | 420 | 230 | 2185 |

### Example 64

### ESC (water) of AAm/BIS hydrogels from kinetic swelling studies

| | % PEG-400 | | | | |
|---|---|---|---|---|---|
| Time(hr) | 7.5 | 12.5 | 17.5 | 22.5 | 27.5 |
| 0.5 | 3.14 | 3.34 | 3.34 | 2.99 | 2.70 |
| 1 | 3.52 | 3.82 | 3.81 | 3.34 | 2.99 |
| 1.5 | 3.81 | 4.14 | 4.18 | 3.59 | 3.23 |
| 2 | 4.05 | 4.44 | 4.48 | 3.81 | 3.42 |
| 3 | 4.47 | 4.97 | 5.10 | 4.20 | 3.76 |
| 4 | 4.86 | 5.55 | 5.50 | 4.54 | 4.05 |
| 5 | 5.23 | 6.03 | 6.04 | 4.93 | 4.42 |
| 24 | 12.04 | 13.16 | 13.02 | 9.3 | 6.84 |
| 48 | 15.22 | 16.40 | 16.53 | 13.21 | 8.58 |

### Example 65

### Preparation of 20%M 2%X AAm/Bis hydrogels using aqueous PEG-400 as solvent

Monomer solutions (10 g) were prepared by dissolving AAm and Bis in the appropriate amount of water and PEG 400 in disposable glass vials. The monomer solution was then degassed by argon purging prior to addition of the initiator system (0.05 mol % initiator per double bond) composed of freshly made up 10% (v/v) TEMED and 10% (w/v) APS. The polymerization was then allowed to proceed at room temperature overnight under an argon environment.

### Example 66

### Optical properties of 20%M 2%X AAm/Bis hydrogels synthesized using aqueous PEG-400 as solvent

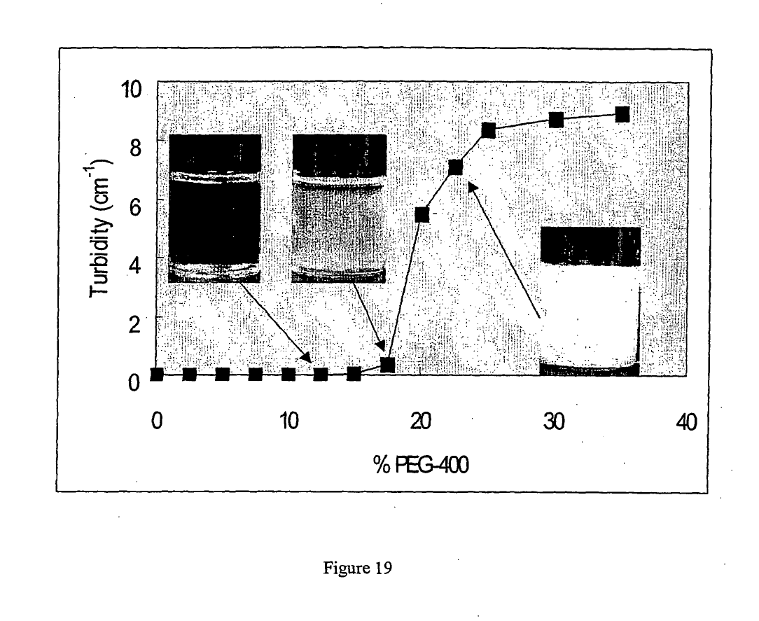

Turbidity results and images of polymers synthesized according to Example 65 are shown in FIG. 19.

### Example 67

### Preparation of optically clear HEMA/EGDMA hydrogels using aqueous propylene glycol as solvent

HEMA hydrogels (10%, 20%, 30%, 40%, 50%, 60%M) were prepared by mixing the appropriate amount of HEMA, EGDMA (1%X, 2%X, 4%X, 6%X, 8%X), propylene glycol and water (10 g total) in disposable glass vials. The monomer solution was then degassed by argon purging for 5 min prior to the addition of the initiator system (0.1 mol % initiator per double bond) composed of freshly made up 10% (w/v) APS and 10% (v/v) TEMED. The polymerization was then allowed to proceed at room temperature overnight under an argon environment.

The propylene glycol content of each reaction mixture was varied in 2.5% (increments from 0%) until an optically clear hydrogel is obtained.

### Example 68

### Critical propylene glycol concentrations for the formation of visually clear HEMA hydrogels at various %M and %X

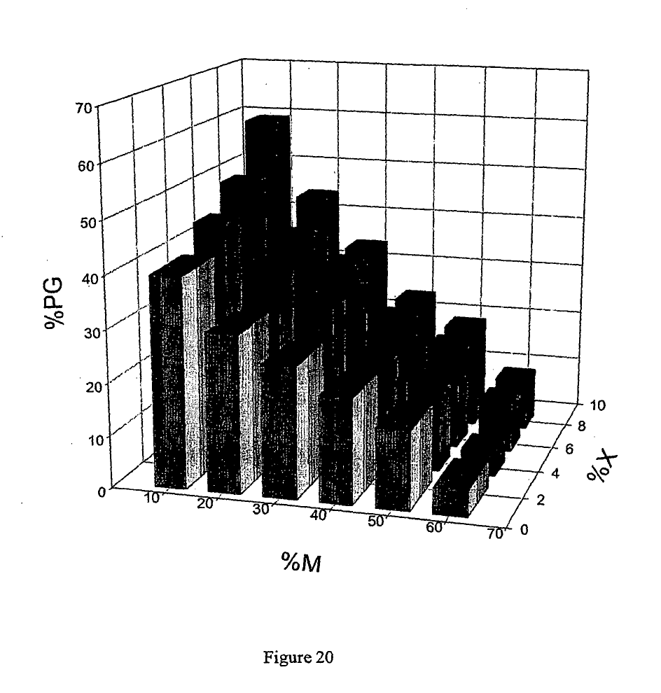

FIG. 20 shows the critical propylene glycol concentrations for the formation of visually hydrogels at various %M and %X.

### Example 69

### Real-time viscosity measurements of 20%M 2%X HEMA polymerizations using aqueous propylene glycol as solvent

Monomer solutions (200 g) were prepared by mixing HEMA and EGDMA in the appropriate amount of water and PG. The monomer solution was then degassed by argon purging prior to addition of the initiator system composed of freshly made up 10% (w/v) APS and 10% (v/v) TEMED (0.1 mol % initiator per double bond). The viscosity of the reaction mixture was measured by a Brookfield® DV-II+ viscometer (0.3 rpm, LV-3 spindle). The experiments were performed in a glove box with controlled oxygen levels (<0.1% O₂).

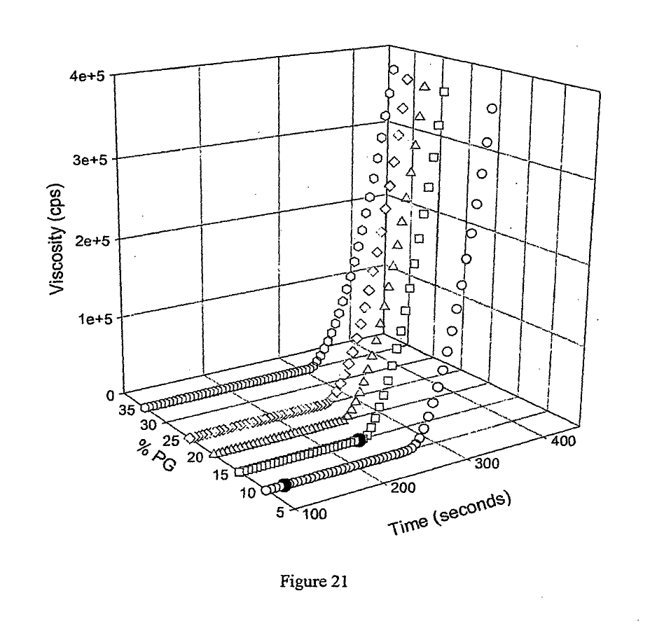

Viscosity measurements of the polymerizations are shown in FIG. 21. Times at which phase separation was observed in the samples are represented by dark coloured points (circle).

Scanning electron microscopy (SEM)

SEM analysis was performed on the hydrogels synthesized in Examples 3 and 4.

After equilibration in water, a piece of hydrogel (5×5 mm) was mounted vertically onto a SEM stub and cryogenically fractured in liquid nitrogen. The water from the fractured surface of the gel was sublimed at 60° C. for 60 min. The gel was then cooled to −190° C. and images of the fractured polymer were taken at 10,000× magnification using a XL30 field emission scanning electron microscope (FESEM).

### Example 70

### SEM analysis of 10%M 2%X AAm/BIS hydrogels synthesized using water, 50% ethylene glycol, or 50% propylene glycol as solvent

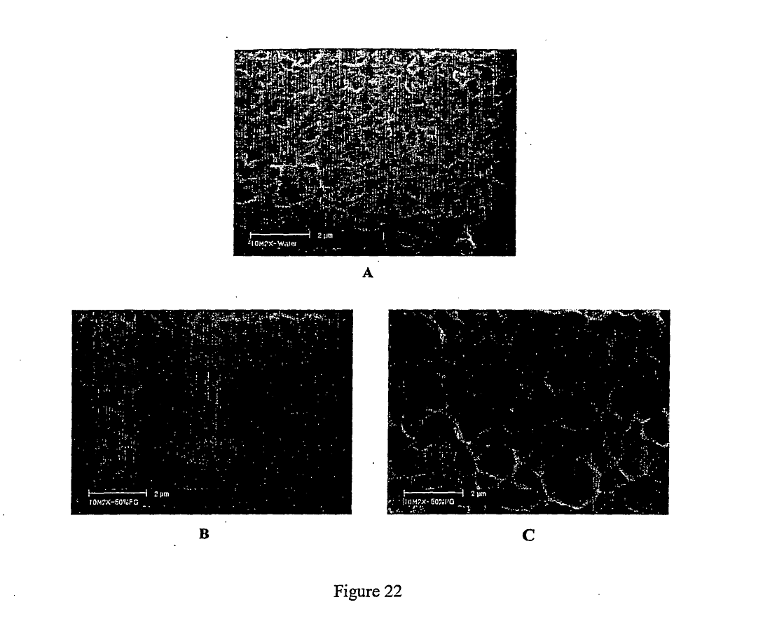

SEM images of the polymers synthesized according to Example 3 and 4 are shown in FIG. 22.

### SUMMARY

Examples 2 to 23 show that the following:

Acrylamide hydrogels can undergo polymerization-induced phase separation when it is synthesized in solvents containing poly(ethylene glycol) with 3 repeating units or more.

Turbidity testing showed that the onset of opacity (i.e. phase separation) occurs at lower concentrations of poly(ethylene glycol) with increasing molecular weight of poly(ethylene glycol).

Acrylamide hydrogels synthesized in the presence of water-soluble entities have in general, larger pores than those synthesized in water. Such gels however cannot be synthesized in solvents containing high concentrations of poly(ethylene glycol) with high molecular weight.

It is well known that when methacrylamide is polymerized in water, an opaque polymer mass is obtained. Example 24 showed that visually clear hydrogels can be obtained from methacrylamide by using hydro-organic solution as the polymerization solvent. Such hydrogels, however, became opaque and lost their mechanical integrity when the organic solvent was subsequently exchanged with water. This demonstrated that although by using a hydro-organic solution as the polymerization solvent, a visually clear hydrogel can be obtained from monomers that produce water-immiscible polymers, many of the resultant hydrogels cannot be used in aqueous media.

Examples 25-27 show that:

HEA hydrogels that are synthesized using water as solvent are opaque and have poor mechanical integrity.

Visually clear HEA hydrogels can be synthesized by careful selection of water-miscible entities. Such gels remained visually clear after the water-miscible entities were exchanged with water. This contrasts with the teaching from prior art observations made in methacrylamide hydrogels.

Examples 28-37 and 42-51 show that:

HEMA hydrogels that are synthesized in water are opaque and have poor mechanical integrity.

Visually clear HEMA hydrogels can be synthesized by careful selection of water-miscible entities. Such gels remained visually clear after the water-miscible entities were exchanged with water.

HEMA hydrogels have very different behaviour to acrylamide hydrogels. Polymerization-induced phase separation occurs at low concentrations of water-miscible entities (e.g. poly(ethylene glycol)), and the gels become more visually clear and the mechanical properties of such gels increases when the concentrations of water-miscible entities increases. This contrasts with prior art acrylamide hydrogels, which state that high concentrations of water-miscible entities would lead to phase separations.

Unexpectedly, turbidity testing shows that in contrast to acrylamide hydrogels, poly(ethylene glycol) with higher molecular weight improves the visual and mechanical properties of the resultant gel (FIG. 5). This contrasts with prior art acrylamide systems, which state that water-miscible entities with high molecular weight would lead to phase separation.

FIG. 7 (Example 31 and 44) shows that visually clear HEMA hydrogels can be obtained from reaction mixtures with low initial monomer concentrations. This contrasts with prior art HEMA gels.

FIGS. 8 and 12 (Example 32, 36 and 45, 49) demonstrate the usage of poly(propylene glycol) as water-miscible entities. The usage of poly(propylene glycol) has not been reported in the literature on hydrogel synthesis.

FIGS. 9 and 10 (Example 33-34 and 46-47) demonstrate the usage of poly(ethylene glycol) derivatives (i.e. alkyl ether) as water-miscible entities. The usage of such derivatives has not been reported in the literature on acrylamide hydrogel synthesis.

FIG. 11 (Example 35 and 48) demonstrate the usage of random and block copolymers of poly(ethylene glycol) and poly(propylene glycol) as water-miscible entities. The usage of such water-miscible entities has not been reported previously.

FIGS. 13 and 14 (Example 36-37 and 49-51) demonstrate the usage of two different types of water-miscible entities together in the same solvent system. The usage of such mixtures of water-miscible entities have not been reported previously.

Example 52 shows that by careful selection of the water-miscible entities, HEMA hydrogels with high water swelling properties can be formed from monomer mixtures with low monomer concentrations (i.e. <50%M). It also shows the increase in water swelling properties with decreasing total monomer concentrations. This contrasts with the prior which states the opposite.

Examples 52 and 53 show that water swelling properties of HEMA hydrogels are dependent upon the initial monomer concentration, the types of water-miscible entities and the concentration of water-miscible entities.

Example 53 further demonstrates that the water swelling properties of the hydrogels increases when the molecular weight of the water-miscible entities (i.e. poly(ethylene glycol) is increased.

Example 54 shows the swelling properties of two different hydrogels. Hydrogel A was synthesized in the presence of a water-miscible entity with low molecular weight; hydrogel B was synthesized in the presence of a water miscible entity with high molecular weight.

Swelling of Hydrogel A and B in mixtures composed of water and organic solvents with different molecular weight shows that:

Hydrogel B swells more in all solvents.

Hydrogel A has low swelling properties in solvents with organic solvents with high molecular weight.

Hydrogel B has significantly higher swellings in solvents with high molecular weight than Hydrogel A.

The above observations show that as the molecular weight of the water-miscible entities increases, the pore size of the gels become dependent upon the size of the water-miscible entities. Such gels have macroporous pores and hence are able to swell more in solvents with high molecular weight solutes, because of the increased diffusion of organic solvent with high molecular weights into the gel.

Examples 56 and 57 demonstrate the usages of poly(ethylene glycol) and poly(propylene glycol) as water-miscible entities in other hydrogels prepared from α,ω-(meth)acryloyloxy monomers. Poly(ethylene glycol) methacrylate was used in these examples. The present invention extends to derivatives of HEMA and HEA, that is, monomers with the same (meth)acrylate ester structure with HEMA and HEA, but different side chains.

Example 58 and 63 show that PIPS occur in 20%M 2%X acrylamide hydrogels synthesized in the presence of 22.5 and 27.5% PEG-400, but can be avoided by the careful selection of the polymerization solvent. It is therefore possible to prepare visually clear hydrogels even when the polymerization solvent is immiscible with the corresponding linear polymer analogues.

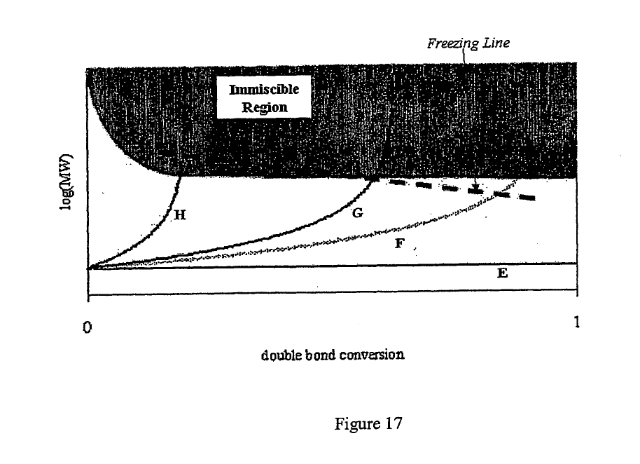

FIG. 17 is a schematic diagram of the formation process of 20%M acrylamide hydrogel, it demonstrates the relationship between the ‘freezing concentration’ of the reaction mixture, the phase boundary, and the concentration and properties of the water-miscible entity which alter the region of immiscibility on the diagram.

Example 59 and FIG. 18 demonstrate the relationship between the ‘freezing concentration’ of the reaction mixture and the phase boundary, it can be seen that visually clear gels can be obtained. In systems where the ‘freezing concentration’ of the reaction mixtures is reached before the onset of PIPS.

Examples 60, 61, and 64 show that hydrogels prepared by the approach of this invention have superior swelling properties to that prepared by systems that reaches the phase boundary before the gel point (22.5 and 27.5% PEG-400).

Example 62 shows that by using a mixture of water and water-miscible entities as the polymerization solvent, visually clear HEMA hydrogels can be prepared even when the polymerization solvent is immiscible with the corresponding linear polymer analogues which are water immiscible.

Examples 65 and 66 show that hydrogels with very different optical properties can be obtained by controlling the ‘freezing point’ of the reaction mixture.

Examples 67 and 68 show that visually clear HEMA hydrogels, at different total monomer concentration and crosslinker concentration, can be synthesized by careful selection of water miscible entities. Such gels remained visually clear after the water-miscible entities were exchanged with water. The critical propylene glycol concentration (and hence critical water content of the reaction mixture) required to obtain a clear gel in these systems are shown in FIG. 20. It can be seen from FIG. 20 that in contrast to the reported values of around 50%, the maximum water content of the reaction mixtures to produce a clear hydrogel is dependent upon both %M and %X. For example, the maximum water content is 30% at 60%M 8%X, and 50% at 10%M 1%X.

Example 69 and FIG. 21 demonstrate the relationship between the ‘freezing concentration’ of the reaction mixture and the phase boundary; it can be seen that visually clear gels can be obtained. In systems where the ‘freezing concentration’ of the reaction mixtures is reached before the onset of PIPS.

Example 70 shows that when compared with Mm hydrogels obtained by existing methods (water as polymerization solvent), hydrogels prepared by the approach of this invention have significantly different pore size and pore size distribution.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A process for producing a polymeric hydrogel having a network containing macropores and micropores comprising:
(a) forming a mixture by adding at least one monomer having at least one double bond, at least one crosslinker having at least two double bonds, an initiation system, and an organic additive to form a hydro-organic system with water; and
(b) allowing the monomer and crosslinker to copolymerize to form a hydrogel having a polymeric network containing macropores and micropores.

2. The process according to claim 1 wherein the monomer having at least one double bond is selected from the group consisting of polyol esters of acrylic acid, polyol esters of methacrylic acid, and mixtures thereof.

3. The process according to claim 1 wherein the monomer having at least one double bond is one or more hydrophilic monomers of polyol esters of acrylic or methacrylic acid.

4. The process according to claim 1 wherein the polyol is selected from the group consisting of polyethylene glycol, polyethylene glycol esters or ethers, polypropylene glycol, polypropylene glycol esters or ethers, random or block copolymers of ethylene glycol and propylene glycol, glycerol, pentaerythritol, ethylene glycol, propylene glycol, and mixtures thereof.

5. The process according to claim 1 wherein the monomer is hydroxyethyl methacrylate (HEMA).

6. The process according to claim 1 wherein the monomer is used at a concentration from 5 to 50%.

7. The process according to claim 1 wherein the crosslinker having at least two double bond is selected from the group consisting of esters of acrylic and/or methacrylic acid, acrylic or methacrylic acid with various polyols, and mixtures thereof.

8. The process according to claim 7 wherein the polyol is selected from the group consisting of polyethylene glycol, polypropylene glycol, random or block copolymers of ethylene glycol and propylene glycol, glycerol, pentaerythritol, ethylene glycol, propylene glycol, and mixtures thereof.

9. The process according to claim 1 wherein the crosslinker is ethylene glycol dimethacrylate (EGDMA).

10. The process according to claim 1 wherein the crosslinker is used at a concentration of greater than about 50% in the mixture of crosslinkers; more preferably greater than about 80%.

11. The process according to claim 1 wherein the polymeric hydrogel is made from a mixture of monomer content of about 10 to 40%M and crosslinker of about 1 to 30%X before polymerization.

12. The process according to claim 11, utilizing HEMA with EGDMA wherein compositions of monomer mixture of HEMA with EGDMA are less than about 40% M and less than about 20% X.

13. The process according to claim 1 wherein a free radical producing method is used as an initiation system.

14. The process according to claim 13 wherein the initiation system is formed by redox, thermal or photo initiators.

15. The process according to claim 14 wherein the redox initiator is formed by ammonium persulphate (APS) with N,N,N′,N′-tetramethylethylenediamine (TEMED).

16. The process according to claim 1 wherein the organic additive is a hydrophilic polymer miscible with water and miscible with a linear polymer produced from the monomer used for copolymerization, or a hydrophilic polymer miscible with water and having a similar solubility parameter (±10(MPa)^{0.5}) to that of a polymer produced from the monomer used for copolymerization.

17. The process according to claim 16 wherein the organic additive is single entity acting as both a porogen to form macropores during the polymerization and a solvent with water to form the hydro-organic solvent.

18. The process according to claim 17 wherein the organic additive is selected from the group consisting of ethylene glycol, polyethylene glycol, propylene glycol, polypropylene glycol, random or block copolymers of ethylene glycol, random or block copolymers of polyethylene glycol, random or block copolymers of propylene glycol, random or block copolymers of polypropylene glycol, ethylene glycol having an ester or ether end group, polyethylene glycol having an ester or ether end group, propylene glycol having an ester or ether end group, polypropylene glycol having an ester or ether end group, and mixtures thereof.

19. The process according to claim 18 wherein the organic additive has the following general formulation: 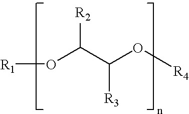
Rd 1, R₄═H, CH₃, —(CH₂)ₓ—CH₃ (x=1-4), —C(═O)—R₅ (R₅═(CH₂)ₓ—CH₃ (x=0-4))
R₂, R₃═H, CH₃, —(CH₂)ₓ—CH₃ (x=1-4), OH

20. The process according to claim 19 wherein the organic additive is a polyethylene glycol or polypropylene glycol.

21. The process according to claim 20 wherein the polyethylene glycol has a molecular weight range from about 100 to about 100,000.

22. The process according to claim 20 wherein the polypropylene glycol has a molecular weight range from about 100 to about 100,000.

23. The process according to claim 16 wherein the organic additive is a copolymer with a hydrophilic component and a hydrophobic component.

24. The process according to claim 23 wherein the organic additive is a copolymer of polyethylene glycol with polypropylene glycol.

25. A polymeric hydrogel having a network containing macropores and micropores produced by the process according to claim 1.

26. A polymeric hydrogel comprising a network of macropores and micropores formed by copolymerizing at least one monomer having at least one double bond and at least one crosslinker having at least two double bonds in the presence of an organic additive forming a hydro-organic system with water.

27. The hydrogel according to claim 26 wherein the monomer having at least one double bond is selected from the group consisting of polyol esters of acrylic acid, polyol esters of methacrylic acid, and mixtures thereof.

28. The hydrogel according to claim 26 wherein the monomer having at least one double bond is one or more hydrophilic monomers from the polyol esters of acrylic or methacrylic acid.

29. The hydrogel according to claim 27 wherein the polyol is selected from the group consisting of polyethylene glycol, polyethylene glycol esters or ethers, polypropylene glycol, polypropylene glycol esters or ethers, random or block copolymers of ethylene glycol and propylene glycol, glycerol, pentaerythritol, ethylene glycol, propylene glycol, and mixtures thereof.

30. The hydrogel according to claim 26 wherein the monomer is hydroxyethyl methacrylate (HEMA).

31. The hydrogel according to claim 26 wherein the monomer is used at a concentration from 5 to 50%.

32. The hydrogel according to claim 26 wherein the crosslinker having at least two double bonds is selected form the group consisting of esters of acrylic and/or methacrylic acid, acrylic or methacrylic acid with various polyols, and mixtures thereof.

33. The hydrogel according to claim 32 wherein the polyol is selected from the group consisting of polyethylene glycol, polypropylene glycol, random or block copolymers of ethylene glycol and propylene glycol, glycerol, pentaerythritol, and ethylene glycol, propylene glycol which are fully or partly esterified, and mixtures thereof.

34. The hydrogel according to claim 26 wherein the crosslinker is ethylene glycol dimethacrylate (EGDMA).

35. The hydrogel according to claim 26 wherein the crosslinker is used at greater than about 50% in the mixture of crosslinkers; more preferably greater than about 80%.

36. The hydrogel according to claim 26 wherein the polymeric hydrogel is made from a mixture of monomer content of 10 to 40%M and crosslinker of 1 to 30%X before polymerization.

37. The hydrogel according to claim 36, wherein compositions of monomer mixture of HEMA with EGDMA are less than about 40% M and less than about 20% X.

38. The hydrogel according to claim 26 wherein a free radical producing method is used as initiation system.

39. The hydrogel according to claim 38 wherein the initiation system is formed by redox, thermal or photo initiators.

40. The hydrogel according to claim 39 wherein the redox initiator is formed by ammonium persulphate (APS) with N,N,N′,N′-tetramethylethylenediamine (TEMED).

41. The hydrogel according to claim 26 wherein the organic additive is a hydrophilic polymer miscible with water and miscible with a linear polymer produced from the monomer used for copolymerization; or a hydrophilic polymer miscible with water and has a similar solubility parameter (±10(MPa)^{0.5}) to that of a polymer produced from the monomer used for copolymerization.

42. The hydrogel according to claim 41 wherein the organic additive is single entity acting as both a porogen to form macropores during the polymerization and a solvent with water to form the hydro-organic solvent.

43. The hydrogel according to claim 42 wherein the organic additive is selected from the group consisting of ethylene glycol, polyethylene glycol, propylene glycol, polypropylene glycol, random or block copolymers of ethylene glycol, random or block copolymers of polyethylene glycol, random or block copolymers of propylene glycol, random or block copolymers of polypropylene glycol, ethylene glycol having an ester or ether end group, polyethylene glycol having an ester or ether end group, propylene glycol having an ester or ether end group, polypropylene glycol having an ester or ether end group, and mixtures thereof.

44. The hydrogel according to claim 43 wherein the organic additive has the following general formulation: 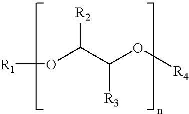
R₁, R₄=H, CH₃, —(CH₂)ₓ—CH₃ (x=1-4), —C(═O)—R₅ (R₅═(CH₂)ₓ—CH₃ (x=0-4))
R₂, R₃=H, CH₃, —(CH₂)ₓ—CH₃ (x=14), OH

45. The hydrogel according to claim 44 wherein the organic additive is a polyethylene glycol or polypropylene glycol.

46. The hydrogel according to claim 45 wherein the polyethylene glycol has a molecular weight range from about 100 to about 100,000.

47. The hydrogel according to claim 45 wherein the polypropylene glycol has a molecular weight range from about 100 to about 100,000.

48. The hydrogel according to claim 41 wherein the organic additive is a copolymer with a hydrophilic component and a hydrophobic component.

49. The hydrogel according to claim 48 wherein the organic additive is a copolymer of polyethylene glycol with polypropylene glycol.

50. The hydrogel according to claim 26 being visually clear.

51. A separation medium formed from the polymeric hydrogel according to claim 25.

52. The separation medium according to claim 51 in the form of a membrane, slab, bead or column.

53. The separation medium according to claim 51 being an electrophoretic medium capable of separating large biomolecules or compounds having a molecular weight of at least 2000 k.

54. A method for separating one or more compounds according to size using electrophoresis, the method comprising:
(a) providing a medium in the form of polymeric hydrogel having a network containing macropores and micropores according to claim 25;
(b) adding one or more compounds to part of the medium; and
(c) applying an electric potential causing at least one compound to pass through the medium, wherein movement through the medium is related to the size of the compound.

55. A size exclusion electrophoresis system comprising:
(a) a cathode;
(b) an anode; and
(c) a separation medium in the form of polymeric hydrogel having a network containing macropores and micropores according to claim 25 capable of separating a mixture of compounds according to size, the medium disposed between the anode and cathode.

56. (canceled)
